(19) United States
(12) Patent Application Publication
(10) Pub. No.: US 2025/0201350 A1
(43) Pub. Date: Jun. 19, 2025

(54) Artificial Intelligence Detection and Treatment of Biological, Chemical and Radiation Exposure

(71) Applicant: CREATIVE MEDICAL TECHNOLOGIES, INC., Phoenix, AZ (US)

(72) Inventors: Thomas Ichim, San Diego, CA (US); Timothy Warbington, Phoenix, AZ (US); Amit Patel, Salt Lake City, UT (US)

(73) Assignee: CREATIVE MEDICAL TECHNOLOGIES, INC., Phoenix, AZ (US)

(21) Appl. No.: 18/982,652

(22) Filed: Dec. 16, 2024

Related U.S. Application Data
(60) Provisional application No. 63/610,556, filed on Dec. 15, 2023.

(51) Int. Cl. G16B 40/30; G16B 20/00

(52) U.S. Cl. CPC G16B 40/30; G16B 20/00

(57) ABSTRACT

Methods, systems, and compositions of matter for detection, analysis and treatment of biological, chemical and radiation exposure using artificial intelligence-based systems including fuzzy logical and/or machine learning. A system capable of compiling physical, biochemical, hematological, psychological and neural datapoints, analyzing said data points and creating actionable therapeutic interventions in a graded manner based on invasiveness and probability of success. Optimized biological intervention to chemical, radiation and biochemical threats using stem cells, T regulatory cells and induced pluripotent stem cells.

### CROSS-REFERENCE TO RELATED APPLICATIONS

The present application claims benefit of U.S. Provisional Application Ser. No. 63/610,556, filed on Dec. 15, 2023, entitled ARTIFICIAL INTELLIGENCE DETECTION AND TREATMENT OF BIOLOGICAL, CHEMICAL AND RADIATION EXPOSURE, the contents of which are incorporated herein by reference in its entirety.

### FIELD OF THE INVENTION

The teachings herein relate to the use of artificial intelligence for detection, analysis and treatment of biological, chemical and radiation exposure.

### BACKGROUND OF THE INVENTION

The use of artificial intelligence (AI) has resulted in fundamental advancements in numerous areas of the medical sciences including genomics, diagnostics, and integrative therapies. The unique ability of AI rapidly acquire information, synthesis Big Data, and provide predictions positions this approach as idea for addressing health care in general and more specifically in emergency settings.

There is a need for government and health care authorities to be alert to illness patterns and diagnostic clues that might indicate a disease outbreak associated with an release of biological or chemical agent, as well as those that occur naturally. It is desirable to quickly identify the type or nature of a chemical or biological event and implement a planned response.

The intentional use of pathological agents such as chemicals, radiation and biological agents against the general population presents a serious risk. Concern about the deliberate use of infectious agents includes anthrax (which can be spread by inhaled spores), small pox, pneumonia, plague, tularemia, and botulism. For example, variola virus, the causative agent of smallpox, is highly infectious and transmitted aerially. While many of these agents have vaccines or treatments, they do not exist in quantities that might be needed and may not be located where an outbreak were to occur. An intentional release of a chemical, radiation or biological agent may take several days or weeks to become apparent. Suspicions may emerge only once patients begin appearing in healthcare facilities or emergency rooms with unusual symptoms or diseases.

Chemical warfare is the intentional use of chemicals to cause disease and death in humans, livestock and crops. Biological warfare is the intentional use of micro-organisms and their toxins to produce disease and death in humans, livestock and crops. The attraction of bioweapons in war, and for use in terrorist attacks is attributed to easy access to a wide range of disease-producing biological agents, to their low production costs, to their non-detection by routine security systems and to their easy transportation from one place to another.

Radiological threats have historically been detected only by direct assessment of energy or particle distribution, which provides little information on a personalized basis for treatment or preventative options.

Identifying biological or chemical agents via lab testing is both time-consuming and costly. There remains a need to develop monitoring and detection systems that can gather and analyze data from multiple locations quickly.

The invention provides means of assessing biological functions and utilizing AI based algorithms to rapidly identify, quantify and provide possible treatments to chemical, radiological and biological threats.

### SUMMARY OF THE INVENTION

A summary is provided below with reference to various numbered aspects of the invention.
1. A method of identifying the potential of developing pathogenic radiological, chemical, or biological reactions using a machine learning system programmed to observe, analyze and predict based on patient derived datapoints.
2. The method of aspect 1, wherein said patient derived datapoint is perturbation in homeostatic functions.
3. The method of aspect 2, wherein said homeostatic functions are selected from a group comprising of: a) blood pressure; b) pulse rate; c) cardiac rhythm; e) electrocardiogram readings; f) electroencephalogram readings; g) near infrared spectroscopy assessment; h) blood gas assessment; i) ocular reflex assessment; j) skin conductance assessment; k) flow mediated dilation assessment; and l) expiratory gas assessment.
4. The method of aspect 1, wherein said patient derived datapoint is perturbation in immunological functions.
5. The method of aspect 4, wherein said immunological function alternation is assessed by levels of immune modulatory proteins in one or more biological matrices.
6. The method of aspect 5, wherein said biological matrix is saliva.
7. The method of aspect 5, wherein said biological matrix is urine.
8. The method of aspect 5, wherein said biological matrix is blood or constituents thereof.
9. The method of aspect 5, wherein said biological matrix is skin.
10. The method of aspect 5, wherein said biological matrix is hair.
11. The method of aspect 5, wherein said biological matrix is sweat.
12. The method of aspect 5, wherein said biological matrix is cerebral spinal fluid.
13. The method of aspect 5, wherein said biological matrix is bronchial fluid.
14. The method of aspect 5, wherein said biological matrix is nail tissue.
15. The method of aspect 5, wherein said immune modulatory protein is an aggregation of proteins.
16. The method of aspect 15, wherein said aggregation of proteins are microvesicles.
17. The method of aspect 15, wherein said aggregation of proteins are exosomes.
18. The method of aspect 15, wherein said aggregation of proteins are apoptotic bodies.
19. The method of aspect 15, wherein said aggregation of proteins are polymers
20. The method of aspect 15, wherein said aggregation of proteins are receptor-ligand complexes.
21. The method of aspect 1, wherein said detection of potential reactions is accomplished by obtaining a biological fluid and irradiating said fluid, for exciting components in the fluid such that at least a portion of the components in the fluid are caused to emit IR radiation in a wavelength range 8-12 μM wavelength; providing a chemical and biological threat detection system having a detector which has observation ability of said biological fluid, for continuously detecting incoming infrared radiation in the wavelength range 8-12 μM wavelength and having an analyzer having reference equations, a reference database, and a computer, for determining quantitative correlations ratios of the unknown chemical biological threat agent that enters the field of monitor of the detector, using direct I0(λ) and I10(λ); and using the computer to determine a ratio of I0(λ) and I10(λ) for comparison with the reference database to detect chemical and biological threats; the quantitative chemical biological IR EM correlations ratios, at various wavelengths (A), being found by correlating with the quantitative low level detection of the change in measured EM energy (8-12 μM wavelength).
22. The method of aspect 21, wherein said biological fluid is a biological matrix.
23. The method of aspect 21, wherein in the step of using the computer, the quantitative chemical biological IR EM correlations ratios are found for three different layers of biological fluid, for the monitored field of view.
24. The method of aspect 23, wherein said biological fluid is extracted at three different parts of the body.
25. The method of aspect 23, wherein said biological fluid is extracted at three different timepoints.
26. The method of aspect 25, wherein said timepoints are separated by at least the time period needed for one circulation of blood throughout the body.
27. The method of aspect 21, wherein in the step of using the computer, the calculations are performed in accordance with Beer-Lamberts law for a single species in a homogeneous media, namely: I(λ′Γ)=I0(λ)e−Lσ(λ)n where I is the measured intensity, L the pathlength in meters, σ(λ) the wavelength dependent absorption cross-sections (m2 molecule−1) and n the number density of the species in units of molecules m−3, and wherein the dimensionless quantity Lσ(λ)n is the optical depth, denoted t.
28. The method of aspect 1, wherein said machine learning system utilizing a knowledge acquisition input platform, connected with a stable computer or cloud residing database, wherein said database comprises a correlation algorithm which scores pathological outcome based on biological parameters.
29. The method of aspect 28, wherein said biological parameters are extracted from existing databases.
30. The method of aspect 28, wherein said biological parameters are extracted from human knowledge.
31. The method of aspect 28, wherein elevation of C reactive protein more than 25% above baseline is utilized to alert need for increased intensity of monitoring.
32. The method of aspect 28, wherein elevation of TNF-alpha more than 25% above baseline is utilized to alert need for increased intensity of monitoring.
33. The method of aspect 28, wherein elevation of HMGB1 more than 25% above baseline is utilized to alert need for increased intensity of monitoring.
34. The method of aspect 28, wherein elevation of interleukin-1 beta more than 25% above baseline is utilized to alert need for increased intensity of monitoring.
35. The method of aspect 28, wherein elevation of interferon gamma more than 25% above baseline is utilized to alert need for increased intensity of monitoring.
36. The method of aspect 28, wherein elevation of interleukin-8 more than 25% above baseline is utilized to alert need for increased intensity of monitoring.
37. The method of aspect 28, wherein elevation of interleukin-12 more than 25% above baseline is utilized to alert need for increased intensity of monitoring.
38. The method of aspect 28, wherein elevation of interleukin-17 more than 25% above baseline is utilized to alert need for increased intensity of monitoring.
39. The method of aspect 28, wherein elevation of interleukin-18 more than 25% above baseline is utilized to alert need for increased intensity of monitoring.
40. The method of aspect 28, wherein one biological parameter of concern is upregulation of innate immune receptor expression on circulating immune cells.
41. The method of aspect 40, wherein said innate immune cell receptor is associated with MyD88.
42. The method of aspect 40, wherein said innate immune cell receptor is associated with MD2.
43. The method of aspect 40, wherein said innate immune cell receptor is toll like receptor 2.
44. The method of aspect 40, wherein said innate immune cell receptor is toll like receptor 3.
45. The method of aspect 40, wherein said innate immune cell receptor is toll like receptor 4.
46. The method of aspect 40, wherein said innate immune cell receptor is toll like receptor 5.
47. The method of aspect 40, wherein said innate immune cell receptor is toll like receptor 7.
48. The method of aspect 40, wherein said innate immune cell receptor is toll like receptor 9.
49. The method of aspect 40, wherein said innate immune cell receptor is toll like MDA.
50. The method of aspect 40, wherein said innate immune cell receptor is NOD1.
51. The method of aspects 40-50, wherein said innate immune cell is a monocyte.
52. The method of aspect 51, wherein said monocyte expresses CD11b.
53. The method of aspect 51, wherein said monocyte expresses CD14.
54. The method of aspect 51, wherein said monocyte expresses CD16.
55. The method of aspect 51, wherein said monocyte expresses CD62 ligand.
56. The method of aspect 51, wherein said monocyte expresses CD115.
57. The method of aspect 51, wherein said monocyte expresses CD163.
58. The method of aspect 51, wherein said monocyte expresses VEGF receptor.
59. A method of utilizing one or more artificial intelligence algorithms to suggest therapeutic dose of hematopoiesis protective therapeutics after exposure to radiation.
60. The method of aspect 59, wherein said radiation is selected from a group comprising of: a) subatomic particles; b) muons; c) proteins; e) alpha rays; f) beta rays; f) x-rays; g) gamma rays; and h) neutrinos.
61. The method of aspect 59, wherein said artificial intelligence algorithm utilizing existing historical data that is used as a training set.
62. The method of aspect 61, wherein said historical data is combined with genotypic, phenotypic and physiological data in respect to the patient being analyzed.
63. The method of aspect 62, wherein gene polymorphisms for interleukin-12 are

evaluated and incorporated into said calculation, wherein enhanced interleukin-12 is associated with enhanced need for mesenchymal stem cell administration.
64. The method of aspect 62, wherein gene polymorphisms for interleukin-18 are evaluated and incorporated into said calculation, wherein enhanced interleukin-18 is associated with enhanced need for mesenchymal stem cell administration.
65. The method of aspect 62, wherein gene polymorphisms for bcl-2Xs are evaluated and incorporated into said calculation, wherein enhanced bcl-2Xs is associated with enhanced need for mesenchymal stem cell administration.
66. The method of aspect 62, wherein gene polymorphisms for TNF-alpha are evaluated and incorporated into said calculation, wherein enhanced TNF-alpha is associated with enhanced need for mesenchymal stem cell administration.
67. The method of aspect 62, wherein gene polymorphisms for lymphotoxin are evaluated and incorporated into said calculation, wherein enhanced lymphotoxin is associated with enhanced need for mesenchymal stem cell administration.
68. The method of aspect 62, wherein gene polymorphisms for TLR4 are evaluated and incorporated into said calculation, wherein enhanced TLR4 is associated with enhanced need for mesenchymal stem cell administration.
69. The method of aspect 62, wherein gene polymorphisms for MyD88 are evaluated and incorporated into said calculation, wherein enhanced MyD88 is associated with enhanced need for mesenchymal stem cell administration.
70. The method of aspect 62, wherein gene polymorphisms for HMGB1 are evaluated and incorporated into said calculation, wherein enhanced HMGB1 is associated with enhanced need for mesenchymal stem cell administration.
71. The method of aspects 63-70, wherein said mesenchymal stem cell is derived from a pluripotent stem cell source.
72. The method of aspect 71, wherein said mesenchymal stem cell is pretreated with one or more agents capable of augmenting expression of interleukin-10.
73. The method of aspect 72, wherein said agent capable of increasing expression of interleukin-10 is hepatocyte growth factor.
74. The method of aspect 72, wherein said agent capable of increasing expression of interleukin-10 is acidic fibroblast growth factor.
75. The method of aspect 72, wherein said agent capable of increasing expression of interleukin-10 is basic fibroblast growth factor.
76. The method of aspect 72, wherein said agent capable of increasing expression of interleukin-10 is ciliary neurotrophic growth factor.
77. The method of aspect 72, wherein said agent capable of increasing expression of interleukin-10 is nerve growth factor.
78. The method of aspect 72, wherein said agent capable of increasing expression of interleukin-10 is brain derived neurotrophic growth factor.
79. The method of aspect 72, wherein said agent capable of increasing expression of interleukin-10 is a TLR4 agonist fibroblast growth factor.
80. The method of aspect 79, wherein said TLR4 agonist is neutrophil extracellular trap concentrate.
81. The method of aspect 79, wherein said TLR4 agonist is OK231.
82. The method of aspect 79, wherein said TLR4 agonist is beta glucan.
83. The method of aspect 79, wherein said TLR4 agonist is hyaluronic acid possessing a molecular weight of less than 5000 Da.
84. The method of aspect 79, wherein said TLR4 agonist is hyaluronic acid possessing a molecular weight of less than 3000 Da.
85. The method of aspect 79, wherein said TLR4 agonist is High Mobility Group Box Protein.
86. The method of aspect 71, wherein said pluripotent stem cell is an induced pluripotent stem cell.
87. The method of aspect 71, wherein said pluripotent stem cell is derived from adipose tissue.
88. The method of aspect 71, wherein said pluripotent stem cell is derived from umbilical cord tissue.
89. The method of aspect 71, wherein said pluripotent stem cell is derived from bone marrow.
90. The method of aspect 71, wherein said pluripotent stem cell is derived from peripheral blood.
91. The method of aspect 71, wherein said pluripotent stem cell is derived from cord blood.
92. The method of aspect 71, wherein said pluripotent stem cell is derived from menstrual blood.
93. The method of aspect 71, wherein said pluripotent stem cell is derived from amniotic fluid.
94. The method of aspect 71, wherein said pluripotent stem cell is derived from amniotic membrane.
95. The method of aspect 71, wherein said pluripotent stem cell is derived from placental tissue.
96. The method of aspect 71, wherein said pluripotent stem cell is derived from skin tissue.
97. The method of aspect 71, wherein said mesenchymal stem cell expresses CD73.
98. The method of aspect 71, wherein said mesenchymal stem cell expresses CD133.
99. The method of aspect 71, wherein said mesenchymal stem cell expresses c-kit.
100. The method of aspect 71, wherein said mesenchymal stem cell expresses c-met.
101. A computer-implemented method for identifying risk of biological, chemical or radiological threat, categorizing potential risk from said threats, and providing means of treatment through the utilization of a artificial intelligence system, said comprising: a) obtaining information regarding physiological characteristics of the individual potentially exposed; b) analyzing one or more molecular and/or cellular features of the individual, wherein analyzing the one or more features includes using an artificial intelligence architecture that includes a separable convolutional neural network; and based at least in part on the analysis of the one or more features; and c) obtaining predictive data in the form of associated risk categorization and potential treatments.
102. The computer-implemented method of aspect 101, which is enabled/designed to create a score for each of the one or more pathologies cellular and molecular features, wherein the score reflects the likelihood that an said biological pathway is providing information and/or detail to reproducibly assess pathology and degree of urgency in which treatment is needed.
103. The computer-implemented method of aspect 101, further comprising indicating whether the one or more molecular and/or biological pathways and/or methodologies have synergy in treatment of the patient
104. The computer-implemented method of aspect 101, further comprising segregating said methodologies and/or molecular pathways into various bioinformatics based categories.
105. The computer implemented method of aspect 105, wherein said bioinformatics based categories include toll like receptor associated pathways.
106. The computer implemented method of aspect 105, wherein said bioinformatics based categories include anthrax receptor associated pathways.
107. The computer implemented method of aspect 1, wherein said therapeutic is a cellular therapeutic, said cellular therapeutic optionally transferred with a pluripotency associated gene.
108. The computer-implemented method of aspect 107, wherein one of the pluripotency associated genes is transfected into said cell to which mechanical stress is to be added.
109. The computer implemented method of aspect 108, wherein said pluripotency associated gene is the octamer-binding transcription factor 4 (OCT4).
110. The computer implemented method of aspect 108, wherein said pluripotency associated gene is the homeobox associated protein NANOG.
111. The computer implemented method of aspect 108, wherein said pluripotency associated gene is the sex determining region Y-box 2 (SOX-2).
112. The computer implemented method of aspect 24, wherein said cell to be converted to a pluripotent stem cell by said mechanical stress associated means is plated in a droplet that partially or fully surrounds said somatic cell.
113. The computer implemented method of aspect 113, wherein forming the droplet includes thermal inkjet printing.
114. The computer implemented method of aspect 112, wherein forming the droplet includes pressurized flow through an orifice without heat.
115. The computer implemented method of aspect 112, wherein forming the droplet includes pressurized flow through an orifice without heat but with increased atmospheric pressure compared to baseline conditions.
116. The computer implemented method of aspect 115, wherein said increased atmospheric pressure is sufficient to induce activation of mitogen activated protein kinase.
117. The computer implemented method of aspect 115, wherein said increased atmospheric pressure is sufficient to induce activation of inhibitor of kappa b.
118. The computer implemented method of aspect 115, wherein said increased atmospheric pressure is sufficient to induce activation of janus activated kinase.
119. The computer implemented method of aspect 115, said increased atmospheric pressure is sufficient to induce activation of NF-kappa B.
120. The computer implemented method of aspect 115, said increased atmospheric pressure is sufficient to induce upregulation of leukemia inhibitory factor receptor.
121. The computer implemented method of aspect 115, wherein said increased atmospheric pressure is sufficient to induce activation of SSEA4.
123. The computer implemented method of aspect 115, wherein said increased atmospheric pressure is sufficient to induce activation of SSEA3.
124. The computer implemented method of aspect 115, wherein said increased atmospheric pressure is sufficient to induce activation of NANOG.
125. The computer implemented method of aspect 115, wherein said increased atmospheric pressure is sufficient to induce activation of c-mpl.
126. The computer implemented method of aspect 115, wherein said increased atmospheric pressure is sufficient to induce activation of TRA-1.
127. The computer implemented method of aspect 115, wherein said increased atmospheric pressure is sufficient to induce activation of c-kit.
128. The computer implemented method of aspect 115, wherein said increased atmospheric pressure is sufficient to induce activation of GDF-9.
129. The computer implemented method of aspect 115, wherein said increased atmospheric pressure is sufficient to induce activation of GDF-11.
130. The computer implemented method of aspect 115, wherein said increased atmospheric pressure is sufficient to induce activation of HDAC6.
131. The computer implemented method of aspect 115, wherein said increased atmospheric pressure is sufficient to induce activation of NOBOX.
132. The computer implemented method of aspect 115, wherein said increased atmospheric pressure is sufficient to induce activation of DAZL.
133. The computer implemented method of aspect 115, wherein said increased atmospheric pressure is sufficient to induce activation of Stella.
134. The computer implemented method of aspect 115, wherein said increased atmospheric pressure is sufficient to increase production of interleukin 1 beta.
135. The computer implemented method of aspect 115, wherein said increased atmospheric pressure is sufficient to increase production of interleukin 1 receptor antagonist.
136. The computer implemented method of aspect 115, wherein said increased atmospheric pressure is sufficient to increase production of interleukin-3.
137. The computer implemented method of aspect 115, wherein said increased atmospheric pressure is sufficient to increase production of interleukin-6.
138. The computer implemented method of aspect 115, wherein said increased atmospheric pressure is sufficient to increase production of interleukin-8.
139. The computer implemented method of aspect 115, wherein said increased atmospheric pressure is sufficient to increase production of interleukin-10.
140. The computer implemented method of aspect 115, wherein said increased atmospheric pressure is sufficient to increase production of G-CSF.
141. The computer implemented method of aspect 115, wherein said increased atmospheric pressure is sufficient to increase production of M-CSF.
142. The computer implemented method of aspect 115, wherein said increased atmospheric pressure is sufficient to increase production of GM-CSF.
143. The computer implemented method of aspect 115, wherein said increased atmospheric pressure is sufficient to increase production of GC-MAF.
144. The computer implemented method of aspect 115, wherein said increased atmospheric pressure is sufficient to increase production of lymphotoxin.
145. The computer implemented method of aspect 115, wherein said increased atmospheric pressure is sufficient to increase production of MCP.
146. The computer implemented method of aspect 115, wherein said increased atmospheric pressure is sufficient to increase production of MIP-18 alpha.
147. The computer implemented method of aspect 115, wherein said increased atmospheric pressure is sufficient to increase production of MIP-1 beta.
148. The computer implemented method of aspect 115, wherein said increased atmospheric pressure is sufficient to increase production of placental growth factor.
149. The computer implemented method of aspect 115, wherein said increased atmospheric pressure is sufficient to increase production of angiopoietin.
150. The computer implemented method of aspect 115, wherein said increased atmospheric pressure is sufficient to increase production of endoglin.
151. The computer implemented method of aspect 115, wherein said increased atmospheric pressure is sufficient to increase production of notch.
152. The computer implemented method of aspect 115, wherein said increased atmospheric pressure is sufficient to increase production of jagged.
153. The computer implemented method of aspect 115, wherein said increased atmospheric pressure is sufficient to increase production of wnt1.
154. The computer implemented method of aspect 115, wherein said increased atmospheric pressure is sufficient to increase production of GDF-11.
155. The computer implemented method of aspect 24, wherein said cell to be converted to a pluripotent stem cell by said mechanical stress associated means is plated in a droplet, wherein forming the droplet includes laser induced fast forward transfer.
156. The computer implemented method of aspect 155, wherein forming the droplet includes using a piezoelectric transducer.
157. The computer implemented method of aspect 155, wherein forming the droplet includes laser bioprinting.
158. The computer implemented method of aspect 155, wherein forming the droplet includes using an ultrasonic transducer.
159. The computer implemented method of aspect 155, wherein forming the droplet includes using at least one microvalve.
160. The computer implemented method of aspect 155, further comprising printing the pluripotent stem cell on a substrate and incubating the pluripotent stem cell.
161. The computer implemented method of aspect 160, wherein said substrate is a decellularized matrix.
162. The computer implemented method of aspect 160, wherein said decellularized matrix is obtained from amniotic membrane.
163. The computer implemented method of aspect 160, wherein said decellularized matrix is obtained from bone marrow.
164. The computer implemented method of aspect 160, wherein said decellularized matrix is obtained from omental tissue.
165. The computer implemented method of aspect 160, wherein said decellularized matrix is obtained from intestinal mucosal tissue.
166. The computer implemented method of aspect 160, wherein said decellularized matrix is obtained from fallopian tube tissue.
167. The computer implemented method of aspect 160, wherein said decellularized matrix is obtained from endometrial tissue.
168. The computer implemented method of aspect 160, wherein said decellularized matrix is obtained from placental tissue.
169. The computer implemented method of aspect 168, wherein said decellularized placental membrane is prepared by obtaining an amniotic sac cut from a placenta around a placenta body, wherein the amniotic sac comprises a pre-decellularized placental membrane and the pre-decellularized placental membrane comprises an amnion layer and a chorion layer.
170. The computer implemented method of aspect 168, wherein the pre-decellularized placental membrane is treated with N-lauroylsarcosinate.
171. The computer implemented method of aspect 168, wherein the pre-decellularized placental membrane is treated with n-octyl-b-D-glucopyranoside.
172. The computer implemented method of aspect 168, wherein the pre-decellularized placental membrane is treated with polyoxyethylene alcohol.
173. The computer implemented method of aspect 168, wherein the pre-decellularized placental membrane is treated with polyoxyethylene isoalcohol.
174. The computer implemented method of aspect 168, wherein the pre-decellularized placental membrane is treated with polyoxyethylene p-t-octyl phenol.
175. The computer implemented method of aspect 168, wherein the pre-decellularized placental membrane is treated with polyoxyethylene nonyphenol.
176. The computer implemented method of aspect 168, wherein the pre-decellularized placental membrane is treated with a or more polyoxyethylene ester of a fatty acid.
177. The computer implemented method of aspect 168, wherein cells are removed from pre-decellularized placental membrane without separating the amnion layer from the chorion layer
178. The computer implemented method of aspect 168, wherein the decellularized placental membrane comprises one or more growth factors in an amount that is at least 10% greater than the sum of the amount of the one or more growth factors in a decellularized control isolated amniotic membrane having the same size as the decellularized placental membrane and the amount of the one or more growth factors in a decellularized control isolated chorionic membrane having the same size as the decellularized placental membrane, wherein the decellularized control isolated amniotic membrane is prepared by removing cells from the amnion layer isolated from the pre-decellularized placental membrane in the same manner as the decellularized placental membrane is prepared from the pre-decellularized placental membrane, and wherein the decellularized control isolated chorion is prepared by removing cells from the chorion layer isolated from the pre-decellularized placental membrane in the same manner as the decellularized placental membrane is prepared from the pre-decellularized placental membrane.
178. The computer implemented method of aspect 168, wherein the decellularized placental membrane comprises one or more growth factors in an amount that is at least 20% of the amount of the one or more growth factors in the pre-decellularized placental membrane.
179. The computer implemented method of aspect 178, wherein said growth factor is HGF-1.
180. The computer implemented method of aspect 178, wherein said growth factor is acidic fibroblast growth factor.
181. The computer implemented method of aspect 178, wherein said growth factor is basic fibroblast growth factor.
182. The computer implemented method of aspect 178, wherein said growth factor is keratinocyte derived growth factor.
183. The computer implemented method of aspect 178, wherein said growth factor is nerve growth factor.
184. The computer implemented method of aspect 178, wherein said growth factor is brain derived neurotrophic growth factor.
185. The computer implemented method of aspect 178, wherein said growth factor is ciliary trophic nerve growth factor.
186. The computer implemented method of aspect 178, wherein said growth factor is transforming growth factor alpha.
187. The computer implemented method of aspect 178, wherein said growth factor is transforming growth factor beta.
188. The computer implemented method of aspect 178, wherein said growth factor is epidermal growth factor.
189. The computer implemented method of aspect 178, wherein said growth factor is vascular endothelial growth factor.
190. The computer implemented method of aspect 178, wherein said growth factor is interleukin-3.
191. The computer implemented method of aspect 178, wherein said growth factor is interleukin-4.
192. The computer implemented method of aspect 178, wherein said growth factor is interleukin-6.
193. The computer implemented method of aspect 178, wherein said growth factor is interleukin-8.
194. The computer implemented method of aspect 178, wherein said growth factor is interleukin-10.
195. The computer implemented method of aspect 178, wherein said growth factor is interleukin-11.
196. The computer implemented method of aspect 178, wherein said growth factor is interleukin-13.
197. The computer implemented method of aspect 178, wherein said growth factor is interleukin-16.
198. The computer implemented method of aspect 178, wherein said growth factor is interleukin-20.
199. The compute implemented method of aspect 178, wherein said growth factor is interleukin-22.
200. The compute implemented method of aspect 178, wherein said growth factor is interleukin-27.
201. The computer implemented method of aspect 168, wherein said decellularized matrix is decellularized placental membrane comprises less than 100 ng dsDNA per mg dry weight of the decellularized placental membrane.
202. The computed implemented method of aspect 168, wherein the decellularized placental membrane comprises DNA in an amount that is less than 10% of the DNA in the pre-decellularized placental membrane.
203. The computed implemented method of aspect 168, wherein the amnion layer in the pre-decellularized placental membrane comprises a fibroblast layer, and wherein the chorion layer in the pre-decellularized placental membrane comprises a reticular layer.
204. The computed implemented method of aspect 168, wherein the amnion layer in the pre-decellularized placental membrane comprises epithelium, a basement membrane, a compact layer, a fibroblast layer, and a spongy layer, and wherein the chorion layer in the pre-decellularized placental membrane comprises a cellular layer, a reticular layer, a pseudo-basement membrane, and a trophoblast layer.
205. The computer implemented method of aspect 168, wherein said placental matrix is treated win a Nobel gas prior to utilization.
206. The computer implemented method of aspect 205, wherein said Nobel gas is xenon.
207. The computer implemented method of aspect 205, wherein said Nobel gas is argon.
208. The computer implemented method of aspect 168, further comprising harvesting the amniotic sac from a donor.
209. The computer implemented method of aspect 168, further comprising cleaning and disinfecting the pre-decellularized placental membrane.
210. The computer implemented method of aspect 168, further comprising fixing the pre-decellularized placental membrane onto a frame before the cell removal step.
211. The computer implemented method of aspect 168, wherein the chorion layer in the pre-decellularized placental membrane comprises a trophoblast layer.
212. The computer implemented method of aspect 168, wherein the one or more compounds are present in the composition at a concentration of 0.01-5% (w/v).
213. The computer implemented method of aspect 168, further comprising treating the pre-decellularized placental membrane with one or more endonucleases at a concentration of 20-400 U/mL.
214. A method of treatment biological, chemical or radiological exposure, wherein said method comprising the steps of: a) selecting a patient who was potentially exposed; b) obtaining physiological, endocrinological, immunological, and regenerative data on said patient; c) providing said data into a machine learning algorithm; and d) utilizing said machine learning algorithm to provide guidance to concentration, frequency and type of cell therapy to be administered.
215. The method of aspect 214, wherein said cell therapy is capable of stimulating hematopoiesis.
216. The method of aspect 215, wherein said agent capable of stimulating hematopoiesis and/or hematopoietic stem cell number and/or activity is progesterone induced blocking factor.
217. The method of aspect 215, wherein said agent capable of stimulating hematopoiesis and/or hematopoietic stem cell number and/or activity is luteinizing hormone.
218. The method of aspect 215, wherein said agent capable of stimulating hematopoiesis and/or hematopoietic stem cell number and/or activity is all trans retinoic acid.
219. The method of aspect 215, wherein said agent capable of stimulating hematopoiesis and/or hematopoietic stem cell number and/or activity is thrombopoietin.
220. The method of aspect 215, wherein said agent capable of stimulating hematopoiesis and/or hematopoietic stem cell number and/or activity is erythropoietin.
221. The method of aspect 215, wherein said agent capable of stimulating hematopoiesis and/or hematopoietic stem cell number and/or activity is monocyte conditioned media.
222. The method of aspect 221, wherein said monocyte conditioned media is generated by culture of monocytes with a toll like receptor agonist.
223. The method of aspect 222, wherein said toll like receptor agonist is a stimulator of TLR2.
224. The method of aspect 223, wherein said TLR2 stimulator is peptidoglycan.
225. The method of aspect 223, wherein said TLR2 stimulator is beta glucan.
226. The method of aspect 222, wherein said toll like receptor agonist is a stimulator of TLR3.
227. The method of aspect 226, wherein said TLR3 stimulator is poly IC.
228. The method of aspect 226, wherein said TLR3 stimulator is poly LCIC.
229. The method of aspect 226, wherein said TLR3 stimulator is double stranded RNA.
230. The method of aspect 226, wherein said TLR3 stimulator is an inactivated RNA virus.
231. The method of aspect 226, wherein said TLR3 stimulator is RGC100.
232. The method of aspect 226, wherein said TLR3 stimulator is ARNAX.
233. The method of aspect 226, wherein said TLR3 stimulator is Poly AU.
234. The method of aspect 222, wherein said toll like receptor agonist is a stimulator of TLR4.
235. The method of aspect 234, wherein said TLR4 stimulator is lipopolysaccharide.
236. The method of aspect 234, wherein said TLR4 stimulator is CRX-527.
237. The method of aspect 234, wherein said TLR4 stimulator is HMGB1.
238. The method of aspect 234, wherein said TLR4 stimulator is MPLA.
239. The method of aspect 234, wherein said TLR4 stimulator is OM-174.
240. The method of aspect 234, wherein said TLR4 stimulator is OM-294.
241. The method of aspect 222, wherein said toll like receptor agonist is a stimulator of TLR5.
242. The method of aspect 241, wherein said stimulator of TLR5 is flagellin.
243. The method of aspect 222, wherein said toll like receptor agonist is a stimulator of TLR7.
244. The method of aspect 243, wherein said stimulator of TLR7 is imiquimod.
245. The method of aspect 222, wherein said toll like receptor agonist is a stimulator of TLR9.
246. The method of aspect 245, wherein said stimulator of TLR9 is ODN2006.
247. The method of aspect 245, wherein said stimulator of TLR9 is ODN2216.
248. The method of aspect 245, wherein said stimulator of TLR9 is ODN2395.
249. The method of aspect 245, wherein said stimulator of TLR9 is CpG DNA.
250. The method of aspect 245, wherein said stimulator of TLR9 is IMO 2055.
251. The method of aspect 245, wherein said stimulator of TLR9 is EMD1201081.
252. The method of aspect 221, wherein said monocyte conditioned media is generated by culture of monocytes under hypoxic conditions while concurrently stimulating one of more TLRs.
253. The method of aspect 221, wherein said monocyte conditioned media is generated by culture of monocytes under hypoxic conditions while concurrently being exposed to IVIG.
254. The method of aspect 253, wherein IVIG is added to said monocytes under hypoxic conditions at a concentration and time point to simulate said monocytes to produce at least 100 ng of interleukin-10 per 1 million monocytes.
255. The method of aspect 253, wherein IVIG is added to said monocytes under hypoxic conditions at a concentration and time point to simulate said monocytes to produce at least 100 ng of interleukin-4 per 1 million monocytes.
256. The method of aspect 253, wherein IVIG is added to said monocytes under hypoxic conditions at a concentration and time point to simulate said monocytes to produce at least 10 ng of G-CSF per 1 million monocytes.
257. The method of aspect 253, wherein IVIG is added to said monocytes under hypoxic conditions at a concentration and time point to simulate said monocytes to produce at least 10 ng of GM-CSF per 1 million monocytes.
258. The method of aspect 253, wherein IVIG is added to said monocytes under hypoxic conditions at a concentration and time point to simulate said monocytes to produce at least 10 ng of M-CSF per 1 million monocytes.
259. The method of aspect 253, wherein IVIG is added to said monocytes under hypoxic conditions at a concentration and time point to simulate said monocytes to produce at least 20 ng of HGF per 1 million monocytes.
260. The method of aspect 253, wherein IVIG is added to said monocytes under hypoxic conditions at a concentration and time point to simulate said monocytes to produce at least 10 ng of IGF-1 per 1 million monocytes.
261. The method of aspect 253, wherein IVIG is added to said monocytes under hypoxic conditions at a concentration and time point to simulate said monocytes to produce at least 10 ng of leukemia inhibitory factor per 1 million monocytes.
262. The method of aspect 253, wherein IVIG is added to said monocytes under hypoxic conditions at a concentration and time point to simulate said monocytes to produce at least 5 ng of vascular endothelial growth factor per 1 million monocytes.
263. The method of aspect 253, wherein IVIG is added to said monocytes under hypoxic conditions at a concentration and time point to simulate said monocytes to produce at least 5 ng of epidermal growth factor per 1 million monocytes.
264. The method of aspect 253, wherein IVIG is added to said monocytes under hypoxic conditions at a concentration and time point to simulate said monocytes to produce at least 15 ng of soluble HLA-G per 1 million monocytes.
265. The method of aspect 253, wherein IVIG is added to said monocytes under hypoxic conditions at a concentration and time point to simulate said monocytes to produce at least 20 ng of soluble TNF-alpha receptor p55 per 1 million monocytes.
266. The method of aspect 253, wherein IVIG is added to said monocytes under hypoxic conditions at a concentration and time point to simulate said monocytes to produce at least 20 ng of soluble TNF-alpha receptor p75 per 1 million monocytes.
267. The method of aspect 253, wherein IVIG is added to said monocytes under hypoxic conditions at a concentration and time point to simulate said monocytes to produce at least 100 ng of interleukin-1 receptor antagonist per 1 million monocytes.
268. The method of aspect 253, wherein IVIG is added to said monocytes under hypoxic conditions at a concentration and time point to simulate said monocytes to produce at least 50 ng of endoglin per 1 million monocytes.
269. The method of aspect 253, wherein IVIG is added to said monocytes under hypoxic conditions at a concentration and time point to simulate said monocytes to produce at least 50 ng of stem cell factor per 1 million monocytes.
270. The method of aspect 253, wherein IVIG is added to said monocytes under hypoxic conditions at a concentration and time point to simulate said monocytes to produce at least 100 ng of GDF-11 per 1 million monocytes.
271. The method of aspect 221, wherein said monocyte conditioned media is generated from monocytes expressing CD14.
272. The method of aspect 221, wherein said monocyte conditioned media is generated from monocytes expressing CD16.
273. The method of aspect 221, wherein said monocyte conditioned media is generated from monocytes expressing CD90.
274. The method of aspect 221, wherein said monocyte conditioned media is generated from monocytes expressing arginase.
275. The method of aspect 221, wherein said monocyte conditioned media is generated from monocytes differentiated from bone marrow.
276. The method of aspect 221, wherein said monocyte conditioned media is generated from monocytes differentiated from peripheral blood progenitors.
277. The method of aspect 276, wherein said peripheral blood progenitors are collected after mobilization.
278. The method of aspect 277, wherein said mobilization is performed by administration of an agent that interferes with the CXCR4-SDF1 axis in the bone marrow.
279. The method of aspect 278, wherein said agent that interferes with said CXCR4-SDF1 axis is mozabil.
280. The method of aspect 278, wherein said agent that interferes with said CXCR4-SDF1 axis is ant antibody to SDF-1.
281. The method of aspect 278, wherein said agent that interferes with said CXCR4-SDF1 axis is an aptamer specific to SDF-1.
282. The method of aspect 278, wherein said agent that interferes with said CXCR4-SDF1 axis is a ribozyme specific to SDF-1.
283. The method of aspect 278, wherein said agent that interferes with said CXCR4-SDF1 axis is an antisense oligonucleotide specific to SDF-1.
284. The method of aspect 278, wherein said agent that interferes with said CXCR4-SDF1 axis is a siRNA specific to SDF-1.
285. The method of aspect 278, wherein said agent that interferes with said CXCR4-SDF1 axis is a shRNA specific to SDF-1.
286. The method of aspect 278, wherein said agent that interferes with said CXCR4-SDF1 axis is an antibody specific to CXCR4.
287. The method of aspect 278, wherein said agent that interferes with said CXCR4-SDF1 axis is an aptamer specific to CXCR4.
288. The method of aspect 278, wherein said agent that interferes with said CXCR4-SDF1 axis is a ribozyme specific to CXCR4.
289. The method of aspect 278, wherein said agent that interferes with said CXCR4-SDF1 axis is a siRNA specific to CXCR4.
290. The method of aspect 278, wherein said agent that interferes with said CXCR4-SDF1 axis is a siRNA specific to CXCR4.
291. The method of aspect 278, wherein said agent that interferes with said CXCR4-SDF1 axis is a shRNA specific to CXCR4.
292. The method of aspect 278, wherein said agent that interferes with said CXCR4-SDF1 axis is a mRNA specific to CXCR4.
293. The method of aspect 278, wherein said agent that interferes with said CXCR4-SDF1 axis is a antisense oligonucleotide specific to CXCR4.
294. The method of aspect 277, wherein said mobilization is induced by treatment of a subject with lipopolysaccharide.
295. The method of aspect 277, wherein said mobilization is induced by treatment of a subject with Poly IC.
296. The method of aspect 277, wherein said mobilization is induced by treatment of a subject with Poly IU.
297. The method of aspect 277, wherein said mobilization is induced by treatment of a subject with neupogen.
298. The method of aspect 277, wherein said mobilization is induced by treatment of a subject with leukine.
299. The method of aspect 277, wherein said mobilization is induced by treatment of a subject with monocyte colony stimulating factor.
300. The method of aspect 277, wherein said mobilization is induced by treatment of a subject with flt3 ligand.
301. The method of aspect 221, wherein said monocyte is generated from a CD34 progenitor cell.
302. The method of aspect 221, wherein said monocyte is generated from a CD133 progenitor cell.
303. The method of aspect 221, wherein said monocyte is generated from a c-kit progenitor cell.
304. The method of aspect 221, wherein said monocyte is generated from a CD127 progenitor cell.
305. The method of aspect 221, wherein said monocyte is extracted from plastic adherent peripheral blood mononuclear cells.
306. The method of aspect 221, wherein said monocyte is extracted from plastic adherent peripheral umbilical cord blood mononuclear cells.
307. The method of aspect 221, wherein said monocyte is extracted from plastic adherent peripheral menstrual blood mononuclear cells.
308. The method of aspect 221, wherein said monocyte is extracted from pluripotent stem cell derived myeloid progenitors.
309. The method of aspect 308, wherein said pluripotent stem cell is an embryonic stem cell.
310. The method of aspect 308, wherein said pluripotent stem cell is a somatic cell nuclear transfer derived stem cell.
311. The method of aspect 308, wherein said pluripotent stem cell is a parthenogenesis derived stem cell.
312. The method of aspect 308, wherein said pluripotent stem cell is an inducible pluripotent derived stem cell.
313. The method of aspect 312, wherein said inducible pluripotent stem cell is generated by retrodifferentiation of somatic cells.
314. The method of aspect 313, wherein said retrodifferentiation is accomplished by transfection with pluripotency associated genes.
315. The method of aspect 313, wherein said retrodifferentiation is accomplished by transfection with pluripotency associated microRNAs.
316. The method of aspect 313, wherein said retrodifferentiation is accomplished by transfection with pluripotency associated mRNA.
317. The method of aspect 313, wherein said retrodifferentiation is accomplished by transfection with pluripotency associated proteins.
318. The method of aspect 314, wherein said pluripotency associated gene is OCT-4.
319. The method of aspect 314, wherein said pluripotency associated gene is KLF.
320. The method of aspect 314, wherein said pluripotency associated gene is Nanog.
321. The method of aspect 314, wherein said pluripotency associated gene is SOX-2.
322. The method of aspect 314, wherein said pluripotency associated gene is k-ras.
323. The method of aspect 314, wherein said pluripotency associated gene is SSEA1.
324. The method of aspect 314, wherein said pluripotency associated gene is TRA-1.
325. The method of aspect 314, wherein said pluripotency associated gene is SSEA2.
326. The method of aspect 314, wherein said pluripotency associated gene is GDF-11 receptor.
327. The method of aspect 312, wherein said pluripotent stem cell is generated by treatment of a somatic cell with one or more pluripotency factors in the presence of a histone deacetylase inhibitor.
328. The method of aspect 327, wherein said histone deacetylase inhibitor is valproic acid.
329. The method of aspect 328, wherein said valproic acid is added to said somatic cells prior to administration of pluripotency genes.
330. The method of aspect 329, wherein said valproic acid is administered to said somatic cells at a concentration and time span sufficient to induce production of at least 10 ng/1 million cells of IL-3.
331. The method of aspect 329, wherein said valproic acid is administered to said somatic cells at a concentration and time span sufficient to induce production of at least 30 ng/1 million cells of IL-6.
332. The method of aspect 329, wherein said valproic acid is administered to said somatic cells at a concentration and time span sufficient to induce production of at least 10 ng/1 million cells of IL-10.
333. The method of aspect 329, wherein said valproic acid is administered to said somatic cells at a concentration and time span sufficient to induce production of at least 50 ng/1 million cells of soluble wnt5.
334. The method of aspect 329, wherein said valproic acid is administered to said somatic cells at a concentration and time span sufficient to induce production of at least 20 ng/1 million cells of soluble HLA-G.
335. The method of aspect 329, wherein said valproic acid is administered to said somatic cells at a concentration and time span sufficient to induce production of at least 30 ng/1 million cells of soluble trem-1.
336. The method of aspect 329, wherein said valproic acid is administered to said somatic cells at a concentration and time span sufficient to induce production of at least 10 ng/1 million cells of soluble ILT3.
337. The method of aspect 329, wherein said valproic acid is administered to said somatic cells at a concentration and time span sufficient to induce production of at least 10 ng/1 million cells of IL-35.
338. The method of aspect 327, wherein said histone deacetylase inhibitor is sodium phenylbutyrate.
339. The method of aspect 338, wherein said sodium phenylbutyrate is administered to said cells in the presence of a GSK-3 inhibitor.
340. The method of aspect 339, wherein said GSK-3 inhibitor is lithium.
341. The method of aspect 339, wherein said lithium is added to said cells together with an activator of TLR4.
342. The method of aspect 342, wherein said TLR4 activator is one or more fragments of lipopolysaccharide.
343. The method of aspect 342, wherein said TLR4 activator is low molecular weight hyaluronic acid.
344. The method of aspect 342, wherein said TLR4 activator is low molecular weight collagen fragments.
345. The method of aspect 342, wherein said TLR4 activator is low molecular weight heparin.
346. The method of aspect 342, wherein said TLR4 activator is low molecular weight syndecan.
347. The method of aspect 342, wherein said TLR4 activator is HMBG1.
348. The method of aspect 342, wherein said TLR4 activator is naltrexone.
349. The method of aspect 342, wherein said TLR4 activator is anti-TLR4 crosslinking antibody.
350. The method of aspect 221, wherein said monocyte conditioned media is generated by culture of monocytes with allogeneic T cells.
351. The method of aspect 350, wherein said T cells are administered to said monocyte cultures in the form of peripheral blood mononuclear cells.
352. The method of aspect 351, wherein said culture of monocytes and peripheral blood mononuclear cells are performed under hypoxia.
353. The method of aspect 351, wherein said culture of monocytes and peripheral blood mononuclear cells are performed under hypoxia in the presence of mesenchymal stem cells.
354. The method of aspect 353, wherein said mesenchymal stem cells are derived from bone marrow.
355. The method of aspect 353, wherein said mesenchymal stem cells are derived from umbilical cord blood.
356. The method of aspect 353, wherein said mesenchymal stem cells are derived from umbilical cord tissue.
357. The method of aspect 353, wherein said mesenchymal stem cells are derived from Wharton's Jelly.
358. The method of aspect 353, wherein said mesenchymal stem cells are derived from fallopian tubes.
359. The method of aspect 353, wherein said mesenchymal stem cells are derived from mobilized peripheral blood.
360. The method of aspect 353, wherein said mesenchymal stem cells are derived omentum.
361. The method of aspect 353, wherein said mesenchymal stem cells are derived from adipose tissue.
362. The method of aspect 361, wherein said adipose tissue is white adipose tissue.
363. The method of aspect 361, wherein said mesenchymal stem cells are derived from brown adipose tissue.
364. The method of aspect 361, wherein said mesenchymal stem cells are derived from pink adipose tissue.
365. The method of aspect 353, wherein said mesenchymal stem cells are derived from deciduous tooth.
366. The method of aspect 353, wherein said mesenchymal stem cells are derived from inducible pluripotent stem cells.
367. The method of aspect 353, wherein said mesenchymal stem cells are derived from parthenogenic stem cells.
368. The method of aspect 353, wherein said mesenchymal stem cells are derived from somatic cell nuclear transfer stem cells.
369. The method of aspect 353, wherein said mesenchymal stem cells are derived from embryonic stem cells
370. The method of aspect 353, wherein said mesenchymal stem cells are derived from very small embryonic like cells.
371. The method of aspect 353, wherein said mesenchymal stem cells express CD90.
372. The method of aspect 353, wherein said mesenchymal stem cells express c-kit.
373. The method of aspect 353, wherein said mesenchymal stem cells express CD44.
374. The method of aspect 353, wherein said mesenchymal stem cells express CD105.
375. The method of aspect 353, wherein said mesenchymal stem cells express PD-L1.
376. The method of aspect 353, wherein said mesenchymal stem cells express PD-L2.
377. The method of aspect 353, wherein said mesenchymal stem cells express FoxP3.
378. The method of aspect 353, wherein said mesenchymal stem cells express AIRE.
379. The method of aspect 353, wherein said mesenchymal stem cells express Fas ligand.
380. The method of aspect 353, wherein said mesenchymal stem cells express CD37.
381. The method of aspect 353, wherein said mesenchymal stem cells express decay accelerating factor.
382. The method of aspect 353, wherein said mesenchymal stem cells express CD73.
383. The method of aspect 353, wherein said mesenchymal stem cells express CD56.
384. The method of aspect 353, wherein said mesenchymal stem cells express CD57.
385. The method of aspect 353, wherein said mesenchymal stem cells express TREM1.
386. The method of aspect 353, wherein said mesenchymal stem cells express TIM-3.
387. The method of aspect 353, wherein said mesenchymal stem cells secrete more than 10 ng of interleukin-10 per million cells upon stimulation with 1 ng lipopolysaccharide/million cells for 24 hours.
388. The method of aspect 353, wherein said mesenchymal stem cells secrete more than 10 ng of interleukin-1 receptor antagonist per million cells upon stimulation with 1 ng lipopolysaccharide/million cells for 24 hours.
389. The method of aspect 353, wherein said mesenchymal stem cells secrete more than 10 ng of TNF-beta per million cells upon stimulation with 1 ng lipopolysaccharide/million cells for 24 hours.
390. The method of aspect 353, wherein said mesenchymal stem cells secrete more than 10 ng of VEGF per million cells upon stimulation with 1 ng lipopolysaccharide/million cells for 24 hours.
391. The method of aspect 353, wherein said mesenchymal stem cells secrete more than 10 ng of EGF per million cells upon stimulation with 1 ng lipopolysaccharide/million cells for 24 hours.
392. The method of aspect 353, wherein said mesenchymal stem cells secrete more than 10 ng of IGF-1 per million cells upon stimulation with 1 ng lipopolysaccharide/million cells for 24 hours.
393. The method of aspect 353, wherein said mesenchymal stem cells secrete more than 10 ng of interleukin-13 per million cells upon stimulation with 1 ng lipopolysaccharide/million cells for 24 hours.
394. The method of aspect 353, wherein said mesenchymal stem cells secrete more than 10 ng of angiopoietin per million cells upon stimulation with 1 ng lipopolysaccharide/million cells for 24 hours.
395. The method of aspect 353, wherein said mesenchymal stem cells secrete more than 10 ng of hepatocyte growth factor per million cells upon stimulation with 1 ng lipopolysaccharide/million cells for 24 hours.
396. The method of aspect 353, wherein said mesenchymal stem cells secrete more than 10 ng of acidic fibroblast growth factor per million cells upon stimulation with 1 ng lipopolysaccharide/million cells for 24 hours.
397. The method of aspect 353, wherein said mesenchymal stem cells secrete more than 10 ng of basic fibroblast growth factor per million cells upon stimulation with 1 ng lipopolysaccharide/million cells for 24 hours.
398. The method of aspect 353, wherein said mesenchymal stem cells secrete more than 10 ng of interleukin-20 per million cells upon stimulation with 1 ng lipopolysaccharide/million cells for 24 hours.
399. The method of aspect 353, wherein said mesenchymal stem cells secrete more than 10 ng of interleukin-22 per million cells upon stimulation with 1 ng lipopolysaccharide/million cells for 24 hours.
400. The method of aspect 353, wherein said mesenchymal stem cells secrete more than 10 ng of interleukin-35 per million cells upon stimulation with 1 ng lipopolysaccharide/million cells for 24 hours.
401. The method of aspect 353, wherein said mesenchymal stem cells secrete more than 10 ng of interleukin-38 per million cells upon stimulation with 1 ng lipopolysaccharide/million cells for 24 hours.
402. The method of aspect 221, wherein said monocytes are cultured in a media generated by an artificial intelligence system whose input is based on patient specific parameters integrating stem cell stimulatory and stem cell inhibitory host characteristics.
403. The method of aspect 221, wherein said monocytes are cultured with T cells in a manner to induce production of at least 20 ng/million cells of hepatocyte growth factor.
404. The method of aspect 403, wherein said T cells are allogeneic to said monocytes.
405. The method of aspect 403, wherein said T cells are autologous to said monocytes.
406. The method of aspect 403, wherein said T cells are xenogeneic to said monocytes.
407. The method of aspect 405, wherein said autologous T cells are activated prior to culture with said monocytes.

### DETAILED DESCRIPTION OF THE INVENTION

The invention provides systems, algorithms, artificial intelligence and machine learning systems capable of obtaining/integrating biological parameters of a subject, assessing possibility of said subject being exposed to chemical, biological or radiological threats, based on this possibility, treatment means of corresponding intensity/invasiveness are provided.

The terms “amplification” or “amplify” or derivatives thereof, as used herein, mean one or more methods known in the art for copying a target or template nucleic acid, thereby increasing the number of copies of a selected nucleic acid sequence. Amplification may be exponential or linear. A “target nucleic acid” refers to a nucleic acid or a portion thereof that is to be amplified, detected, and/or sequenced. A target or template nucleic acid may be any nucleic acid, including DNA or RNA. A target nucleic acid may be characteristic of a biothreat, also referred to herein as a “biothreat pathogen target nucleic acid.” Exemplary, non-limiting biothreat pathogen target nucleic acids include target nucleic acids characteristic of Bacillus anthracis (e.g., Bacillus anthracis pX01 plasmid and/or Bacillus anthracis pX02 plasmid), Francisella tularensis, Burkholderia spp., Yersinia pestis, and/or Rickettsia prowazekii. A target nucleic acid may include a drug resistance marker (e.g., a drug resistance gene such as an antibiotic resistance gene, e.g., an antibiotic resistance gene selected from the group consisting of KPC, NDM, VIM, IMP, OXA-48-like, CMY, CTX-M 14, CTX-M 15, Qnr, Tet, otr, tcr3, qepAB, opxAB, gyrA, gyrB, and parC) or a portion thereof that is to be amplified, detected, and/or sequenced. The sequences amplified in this manner form an “amplified target nucleic acid,” “amplified region,” or “amplicon,” which are used interchangeably herein. Primers and/or probes can be readily designed to target a specific template nucleic acid sequence. Exemplary amplification approaches include but are not limited to polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, and ramification amplification (RAM).

The term “drug resistance” refers to the ability of a pathogen (e.g., a biothreat pathogen, including an engineered biothreat pathogen) to resist one or more effects of a therapeutic agent. For example, “antimicrobial resistance” refers to the ability of a microbe (e.g., a bacterial or fungal pathogen) to resist one or more effects of an antimicrobial agent, and “antibiotic resistance” refers to the ability of a bacterium to resist one or more effects of an antibiotic agent. Drug-resistant pathogens can be more difficult to treat than drug-sensitive pathogens. Resistance can occur naturally in pathogens, or can arise via spontaneous mutation or by gene transfer between different species. A pathogen may be become resistant to a therapeutic agent that previously was able to treat an infection caused by the pathogen. In some embodiments, a drug-resistant pathogen is able to survive or proliferate upon exposure to a concentration of a therapeutic agent that would kill or slow proliferation of a drug-sensitive pathogen. An “antibiotic resistance gene” or an “antibiotic resistance target nucleic acid” refers to a gene that confers or facilitates antibiotic resistance, or a portion thereof. Exemplary antibiotic (e.g., carbapenem) resistance genes include, but are not limited to, KPC, NDM, VIM, IMP, OXA-48-like, CMY, CTX-M 14, CTX-M 15, Qnr, Tet, otr, tcr3, qepAB, opxAB, gyrA, gyrB, and parC. Additional antibiotic resistance genes are described herein or are known in the art. In the literature, the enzymes encoded by these genes are typically spelled in capital letters, while the gene names are italicized. For example, the enzyme NDM is encoded by the blaNDM gene. This convention generally holds for all of the beta lactamase genes (e.g., NDM, KPC, IMP, VIM, DHA, CMY, FOX, CTX-M, SHV, TEM, and OXA-48-like).

The term “sequencing” refers to any method for determining the nucleotide order of a nucleic acid (e.g., DNA), such as a target nucleic acid or an amplified target nucleic acid. Exemplary sequencing approaches include but are not limited to massively parallel sequencing (e.g., sequencing by synthesis (e.g., ILLUMINA™ dye sequencing, ion semiconductor sequencing, or pyrosequencing) or sequencing by ligation (e.g., oligonucleotide ligation and detection (SOLID™) sequencing or polony-based sequencing)), long-read or single-molecule sequencing (e.g., Helicos™ sequencing, single-molecule real-time (SMRT™) sequencing, and nanopore sequencing) and Sanger sequencing. Massively parallel sequencing is also referred to in the art as next-generation or second-generation sequencing, and typically involves parallel sequencing of a large number (e.g., thousands, millions, or billions) of spatially-separated, clonally amplified templates or single nucleic acid molecules. Short reads are often used in massively parallel sequencing. See, e.g., Metzker, Nature Reviews Genetics 11:31-36, 2010. Long-read sequencing and/or single-molecule sequencing are sometimes referred to as third-generation sequencing. Hybrid approaches (e.g., massively parallel and single molecule approaches or massively parallel and long-read approaches) can also be used. It is to be understood that some approaches may fall into more than one category, for example, some approaches may be considered both second-generation and third-generation approaches, and some sources refer to both second and third generation sequencing as “next-generation” sequencing.

By “analyte” is meant a substance or a constituent of a sample to be analyzed. Exemplary analytes include one or more species of one or more of the following: a nucleic acid (e.g., DNA or RNA (e.g., mRNA)), an oligonucleotide, a protein, a peptide, a polypeptide, an amino acid, an antibody, a carbohydrate, a polysaccharide, glucose, a lipid, a gas (e.g., oxygen or carbon dioxide), an electrolyte (e.g., sodium, potassium, chloride, bicarbonate, blood urea nitrogen (BUN), magnesium, phosphate, calcium, ammonia, lactate), a lipoprotein, cholesterol, a fatty acid, a glycoprotein, a proteoglycan, a lipopolysaccharide, a cell surface marker (e.g., a cell surface protein of a pathogen), a cytoplasmic marker (e.g., CD4/CD8 or CD4/viral load), a therapeutic agent, a metabolite of a therapeutic agent, a marker for the detection of a weapon (e.g., a chemical or biological weapon), an organism, a pathogen, a pathogen byproduct, a parasite (e.g., a protozoan or a helminth), a protist, a fungus (e.g., yeast (e.g., a Candida species (e.g., Candida albicans, Candida glabrata, Candida krusei, C. parapsilosis, Candida auris, Candida lusitaniae, Candida haemulonii, Candida duobushaemulonii, Candida pseudohaemulonii, Candida guilliermondii, and C. tropicalis)) or mold), a bacterium (e.g., Acinetobacter baumannii, Escherichia coli, Enterococcus faecalis, Enterococcus faecium, Klebsiella pneumoniae, Pseudomonas aeruginosa, Staphylococcus aureus, Borrelia burgdorferi, Borrelia afzelii, Borrelia garinii, Rickettsia rickettsii, Anaplasma phagocytophilum, Coxiella burnetii, Ehrlichia chaffeensis, Ehrlichia ewingii, Francisella tularensis, Streptococcus pneumoniae, Enterobacter cloacae, Streptococcus pyogenes, Streptococcus mutans, Streptococcus sanguinis, Haemophilus influenzae, or Neisseria meningitides), an actinomycete, a cell (e.g., a whole cell, a tumor cell, a stem cell, a white blood cell, a T cell (e.g., displaying CD3, CD4, CD8, IL2R, CD35, or other surface markers), or another cell identified with one or more specific markers), a virus (e.g., a coronavirus (e.g., a SARS-CoV or a SARS-CoV-2), a prion, a plant component, a plant by-product, algae, an algae by-product, plant growth hormone, an insecticide, a man-made toxin, an environmental toxin, an oil component, and components derived therefrom. In particular embodiments, the analyte is a nucleic acid (e.g., DNA or RNA (e.g., mRNA)), such as a target nucleic acid or an amplified target nucleic acid. In some embodiments, the analyte is a biothreat pathogen target nucleic acid (e.g., a target nucleic acid characteristic of Bacillus anthracis (e.g., Bacillus anthracis pX01 plasmid and/or Bacillus anthracis pX02 plasmid), Francisella tularensis, Burkholderia spp., Yersinia pestis, and/or Rickettsia prowazekii). In further particular embodiments, the analyte is a drug resistance marker (e.g., an antibiotic resistance gene, e.g., an antibiotic resistance gene selected from the group consisting of KPC, NDM, VIM, IMP, OXA-48-like, CMY, CTX-M 14, CTX-M 15, Qnr, Tet, otr, tcr3, qepAB, opxAB, gyrA, gyrB, and parC) or a portion thereof.

A “biological sample” is a sample obtained from a subject including but not limited to blood (e.g., whole blood, processed whole blood (e.g., a crude whole blood lysate), serum, plasma, and other blood derivatives), bloody fluids (e.g., wound exudate, phlegm, bile, and the like), urine, cerebrospinal fluid (CSF), synovial fluid (SF), breast milk, sweat, tears, saliva, semen, feces, vaginal fluid or tissue, sputum (e.g., purulent sputum and bloody sputum), nasopharyngeal aspirate or swab, lacrimal fluid, mucous, epithelial swab (e.g., a buccal swab, an axilla swab, a groin swab, an axilla/groin swab, or an ear swab), tissues (e.g., tissue biopsies (e.g., skin biopsies (e.g., from wounds, burns, or tick bites), muscle biopsies, or lymph node biopsies)), including tissue homogenates), organs, bones, teeth, or culture media (e.g., BHI, SABHI, SDA, LB, and the like), among others. In some embodiments, the biological sample is whole blood, which may contain an anticoagulant (e.g., EDTA, sodium citrate, sodium heparin, lithium heparin, and/or potassium oxylate/sodium fluoride). In several embodiments, the biological sample contains cells, cell debris, and/or nucleic acids (e.g., DNA) derived from the subject from which the sample was obtained. In particular embodiments, the subject is a host of a pathogen (e.g., a biothreat pathogen), and the biological sample obtained from the subject includes subject (host)-derived cells, cell debris, and nucleic acids (e.g., DNA), as well as one or more pathogen cells. The biological sample may be a swab sample, which may include a swab buffer diluent or swab transport medium. In some embodiments, the swab buffer diluent or swab transport medium is, without limitation, PBST, Amies Buffer, Amies Buffer+10% (v/v) 10×PBST, Cary Blair Media, or Liquid Stuart Swabs (which may include addition of 10% (v/v) 10×PBST). The biological sample may be a liquid sample.

The term “biomarker” is a biological substance that can be used as an indicator of a particular disease state or particular physiological state of an organism, generally a biomarker is a protein or other native compound measured in bodily fluid whose concentration reflects the presence or severity or staging of a disease state or dysfunction, can be used to monitor therapeutic progress of treatment of a disease or disorder or dysfunction, or can be used as a surrogate measure of clinical outcome or progression. In some embodiments, the biomarker is a nucleic acid (e.g., DNA or RNA (e.g., mRNA)).

A “pathogen” means an agent causing disease or illness to its host, such as an organism or infectious particle, capable of producing a disease in another organism, and includes but is not limited to bacteria (e.g., Acinetobacter baumannii, Escherichia coli, Enterococcus faecalis, Enterococcus faecium, Klebsiella pneumoniae, Pseudomonas aeruginosa, Staphylococcus aureus, Borrelia burgdorferi, Borrelia afzelii, Borrelia garinii, Rickettsia rickettsii, Anaplasma phagocytophilum, Coxiella burnetii, Ehrlichia chaffeensis, Ehrlichia ewingii, Francisella tularensis, Streptococcus pneumoniae, Enterobacter cloacae, Streptococcus pyogenes, Streptococcus mutans, Streptococcus sanguinis, Haemophilus influenzae, or Neisseria meningitides), viruses (e.g., any virus disclosed herein, including a coronavirus (e.g., SARS-COV or SARS-COV-2), protozoa, prions, fungi (e.g., yeast (e.g., Candida species), or pathogen by-products. “Pathogen by-products” are those biological substances arising from the pathogen that can be deleterious to the host or stimulate an excessive host immune response, for example pathogen nucleic acids, antigen(s), metabolic substances, enzymes, biological substances, or toxins (e.g., Bacillus anthracis toxin genes protective antigen (pagA), edema factor (cya), and lethal factor (lef); enteropathogenic E. coli translocated intimin receptor (Tir); Clostridium difficile toxins TcdA and TcdB; and Clostridium botulinum toxins BoNT/A, BoNT/B, BoNT/C, BoNT/D, BONT/E, BONT/F, and BoNT/G). In some embodiments, the pathogen is a bacterial pathogen, e.g., a drug resistant bacterial pathogen, e.g., a bacterial pathogen that expresses one or more antibiotic resistance genes selected from the group consisting of KPC, NDM, VIM, IMP, OXA-48-like, CMY, CTX-M 14, CTX-M 15, Qnr, Tet, otr, tcr3, qepAB, opxAB, gyrA, gyrB, and parC. In several embodiments, the pathogen is a biothreat pathogen.

A “biothreat pathogen” refers to any pathogen that poses a threat to health. A biothreat pathogen may be naturally occurring or engineered. A biothreat pathogen may be used in bioterrorism or biological warfare. In other instances, a biothreat pathogen may be involved in an outbreak or epidemic. The CDC and NIAID, in conjunction with the U.S. Department of Homeland Security, evaluate the potential threat from various microorganisms and toxins and classify them into three categories. Category A includes agents that are considered to represent the highest risk, including Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers (e.g., Arenaviruses (e.g., Junin, Machupo, Guanarito, Chapare, Lassa, and Lujo), Bunyaviruses (e.g., Hantaviruses causing Hanta pulmonary syndrome, Rift Valley Fever, and Crimean Congo Hemorrhagic Fever), Flaviviruses (e.g., Dengue), and Filoviruses (e.g., Ebola and Marburg viruses). Category B agents could conceivably threaten water and food safety and include Burkholderia pseudomallei (melioidosis), Coxiella burnetii (Q fever), Brucella species (spp.) (brucellosis), Burkholderia mallei (glanders), Chlamydia psittaci (Psittacosis), Ricin toxin (Ricinus communis), Epsilon toxin (Clostridium perfringens), Staphylococcus enterotoxin B (SEB), Typhus fever (Rickettsia prowazekii), food and waterborne pathogens (e.g., bacteria (e.g., diarrheagenic E. coli, pathogenic Vibrios, Shigella spp., Salmonella spp., Listeria monocytogenes, Campylobacter jejuni, and Yersinia enterocolitica), viruses (e.g., Caliciviruses and Hepatitis A), protozoa (e.g., Cryptosporidium parvum, Cyclospora cayatanensis, Giardia lamblia, Entamoeba histolytica, Toxoplasma gondii, Naegleria fowleri, Balamuthia mandrillaris), and fungi (e.g., Microsporidia)), and mosquito-borne viruses (e.g., West Nile virus, LaCross encephalitis, California encephalitis, Venezuelan equine encephalitis, Eastern equine encephalitis, Japanese encephalitis virus, St. Louis encephalitis virus, Yellow fever virus, Chikungunya virus, and Zika virus). Category C agents are considered emerging infectious disease threats which could be engineered for mass dissemination, including Nipah and Hendra viruses, Additional hantaviruses, Tickborne hemorrhagic fever viruses (e.g., Bunyaviruses (e.g., Severe Fever with Thrombocytopenia Syndrome virus (SFTSV), Heartland virus) and Flaviviruses (e.g., Omsk Hemorrhagic Fever virus, Alkhurma virus, Kyasanur Forest virus)), tickborne encephalitis complex flaviviruses (e.g., Tickborne encephalitis viruses, Eastern subtype, Far Eastern subtype, Siberian subtype, and Powassan/Deer Tick virus), tuberculosis (e.g., drug-resistant tuberculosis), influenza virus, other Rickettsias, Rabies virus, prions, Coccidioides spp., severe acute respiratory syndrome associated coronavirus (SARS-CoV), MERS-CoV, SARS-CoV-2 (which causes COVID-19), and other highly pathogenic human coronaviruses, and antimicrobial resistance. In particular embodiments, the biothreat pathogen is selected from the group consisting of Bacillus anthracis, Francisella tularensis, Burkholderia spp. (e.g., B. mallei or B. pseudomallei), Yersinia pestis, and Rickettsia prowazekii.

In one embodiment, the invention provides means of assessing risk associated with biological and/or radiological and/or chemical pathological risks. In one specific embodiment the invention relates to a chemical biological (CB) defense technology solution for enhancing the discrimination in chemical biological threat detection in blood or other biological fluids. The invention uses the concept of quantitative monitoring of the atmospheric air for chemical biological threats, as well as measuring the quantitative correlations/ratios in differential changes in the air, for the active/passive chemical biological Infrared (IR) EM radiation at an 8-12 μM wavelength, absorption/emission/scattering, using Electro-Optics. In one embodiment the invention provides means of combating the weapons of mass destruction. chemical biological (CB) defense programs. The chemical or biological threats are of many types, and this term will be understood with reference to the prior art. Defense technology solutions for tactical improvements of chemical biological threat detection of this invention detect electro-magnetic radiation (photons) of 8-12 μM wavelength. The electro-optics include enhancements in discrimination for detecting minimum detectable chemical biological agent concentrations in COB threat detection, by the use of quantitative correlations/ratios of chemical biological electro-magnetic (EM) radiation (photons), in the range of 8-12 μM wavelengths. This includes use of electro-optics. In one embodiment, bacillus subtilis bacteria and Kaolin interference have been chosen for comparison, for demonstrating this defense technology solution. Dealing with the chemical biological threat detection issues, some basic relationships and concepts concerning electro-magnetic (EM) theory and radioactive processes are also important. The part of the electromagnetic spectrum that is important in remote sensing covers wavelengths of the order 0.1 μm (UV) to 100 m (Radio HF). For wavelengths of 8-12 μm, the atmosphere is more transparent with structures from absorbing species. For dealing with standoff chemical biological detection issues, some basic relationships and concepts concerning electro-magnetic (EM) and radioactive processes are also important. It is important that the distinction is clear between the issue of chemical biological threat detection and the instrument detectability of the chemical biological threat. Although the two are related to some extent, they are different in many respects. The instrument detectability issue relates more to the methodologies adopted by the instruments in use, while the overall threat chemical biological detection is not just a factor of the instrument factors, but is part of an overall chemical biological threat detection strategy.

In another embodiment the invention provides means of assessing potential exposure based on artificial intelligence systems, which provide a degree of risk of exposure. Once a risk of exposure is ascertained, definitive tests may be conducted. For example, methods such as those described by Snyder et al in US patent application #20220372555 may be used. In one embodiment a system for the detection of one or more biothreat pathogen target nucleic acids, the system including: (a) a first unit including (i) a permanent magnet defining a magnetic field; (ii) a support defining a well holding a liquid sample including magnetic particles having a mean particle diameter between 700 and 1200 nm, preferably between 650 and 950 nm, and one or more biothreat pathogen target nucleic acids characteristic of Bacillus anthracis pX01 plasmid, Bacillus anthracis pX02 plasmid, Francisella tularensis, Burkholderia spp., Yersinia pestis, and/or Rickettsia prowazekii, and having an RF coil disposed about the well, the RF coil configured to detect a signal produced by exposing the liquid sample to a bias magnetic field created using the permanent magnet and an RF pulse sequence; and (iii) one or more electrical elements in communication with the RF coil, the electrical elements configured to amplify, rectify, transmit, and/or digitize the signal; and (b) a second unit including a removable cartridge sized to facilitate insertion into and removal from the system, wherein the removable cartridge is a modular cartridge including (i) a reagent module for holding one or more assay reagents, (ii) a detection module including a detection chamber for holding a liquid sample including the magnetic particles and the one or more analytes, and, optionally, (iii) a sterilizable inlet module, wherein the reagent module, the detection module, and, optionally, the sterilizable inlet module, can be assembled into the modular cartridge prior to use, and wherein the detection chamber is removable from the modular cartridge, preferably, wherein the system further includes a system computer with processor for implementing an assay protocol and storing assay data, and wherein the removable cartridge further includes (i) a readable label indicating the analyte to be detected, (ii) a readable label indicating the assay protocol to be implemented, (iii) a readable label indicating a patient identification number, (iv) a readable label indicating the position of assay reagents contained in the cartridge, or (v) a readable label including instructions for the programmable processor.

In some embodiments, detection of biothreat pathogen target nucleic acids allows for rapid, accurate, and high sensitivity detection and identification of a biothreat pathogen present in a biological or environmental sample containing host cells, cell debris, and/or host cell nucleic acids (e.g., DNA), including but not limited to whole blood, processed whole blood (e.g., a crude whole blood lysate), serum, plasma, or other blood derivatives; bloody fluids such as wound exudate, phlegm, bile, and the like; bronchiolar lavage (BAL), urine, tissue samples (e.g., tissue biopsies); and sputum (e.g., purulent sputum and bloody sputum)), which may be used, for example, for diagnosis of a pathogen exposure (e.g., sepsis, bloodstream infections (BSIs) (e.g., bacteremia, fungemia (e.g., Candidemia), and viremia), anthrax, botulism, plague, tularemia, viral hemorrhagic fevers, melioidosis, Q fever, brucellosis, glanders, Psittacosis, tickborne hemorrhagic fever viruses Lyme disease, septic shock, and diseases that may manifest with similar symptoms to diseases caused by or associated with biothreat pathogens, e.g., systemic inflammatory response syndrome (SIRS)). Treatment means contemplated by the invention are determined by AI-based analysis which provides indications as to type of therapy, concentration of therapy, and dosage of therapy. In some embodiments stem cells are utilized therapeutic. Said stem cells may include stem cells generated from pluripotent stem cells, and/or stem cells generated from adult sources. Said stem cells may be utilized for suppression of radioactive adverse effects, as well as reduction of inflammation. In some embodiments stem cells are utilized to reduce hematopoietic toxicity of radioactive exposure.

In one embodiment the invention provides a system for designing a treatment or prophylactic plan to reverse or prevent pathology associated with biological, chemical or radiological threat, wherein said system uses as a component or as a backbone a machine learning artificial intelligence paradigm. In one embodiment, the system disclosed includes at least a server, wherein said server is designed and configured to receive training data, wherein receiving the training data further comprises receiving a first training set including a plurality of first data entries, each first data entry of the plurality of first data entries including at least an element of physiological state data and at least a correlated first prognostic label; and receiving a second training set including a plurality of second data entries, each second data entry of the plurality of second data entries including at least a second prognostic label and at least a correlated ameliorative process label. The system includes a diagnostic engine operating on the at least a server, wherein the diagnostic engine is configured to record at least a biological extraction from a user. The diagnostic engine operating on the at least a server is further configured to generate a diagnostic output based on the at least a biological extraction and the training data, wherein generating further comprises performing at least a machine-learning algorithm as a function of the training data and the at least a biological extraction. The system includes a plan generator module operating on the at least a server, the plan generator module designed and configured to generate, a comprehensive instruction set associated with the user as a function of the diagnostic output. The system includes a supplement plan generation module operating on the at least a server, the supplement plan generation module designed and configured to generate, a supplement instruction set associated with the user as a function of the comprehensive instruction set.

For the practice of the invention, training data may include one or more elements that are not categorized; that is, training data may not be formatted or contain descriptors for some elements of data. Machine-learning algorithms and/or other processes may sort training data according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name and/or a description of a medical condition or therapy may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms.

For the purpose of the invention, said physiological state data may include, without limitation, hematological data, such as red blood cell count, which may include a total number of red blood cells in a person's blood and/or in a blood sample, hemoglobin levels, hematocrit representing a percentage of blood in a person and/or sample that is composed of red blood cells, mean corpuscular volume, which may be an estimate of the average red blood cell size, mean corpuscular hemoglobin, which may measure average weight of hemoglobin per red blood cell, mean corpuscular hemoglobin concentration, which may measure an average concentration of hemoglobin in red blood cells, platelet count, mean platelet volume which may measure the average size of platelets, red blood cell distribution width, which measures variation in red blood cell size, absolute neutrophils, which measures the number of neutrophil white blood cells, absolute quantities of lymphocytes such as B-cells, T-cells, Natural Killer Cells, and the like. In some embodiments specific subsets of lymphocytes may be quantified. Said quantification of lymphocytes may be performed based on size, density, surface markers, biological activity, cytokine production and homing activity. In some embodiments T cells are assessed for cytotoxic activity. In other embodiments production of cytokines from T cells is quantified. Notable cytokines include ABCF1, BCL6, C3, C4A, CEBPB, CRP, ICEBERG, IL1R1, IL1RN, IL8RB, LTB4R, TOLLIP, IFNA2, IL10RA, IL10RB, IL13, IL13RA1, IL5RA, IL9, IL9R, CD40LG (TNFSF5), IFNA2,, IL17C, IL1A, IL1B, IL1F10, IL1F5, IL1F6, IL1F7, IL1F8, IL1F9, IL22, IL5, IL-6, IL8, IL9, IL-18, IL-33, LTA, LTB, MIF, SCYE1, SPP1, TNF, CCL13 (mcp-4), CCR1, CCR2, CCR3, CCR4, CCR5, CCR6, CCR7, CCR8, CCR9, CX3CR1, IL8RA, XCR1 (CCXCR1), C5, CCL1 (1-309), CCL11 (eotaxin), HMGB1, IL-2. IL-12, IL-17, IL33. CCL13 (mcp-4), CCL15 (MIP-1d), CCL16 (HCC-4), CCL17 (TARC), CCL18 (PARC), CCL19, CCL2 (mcp-1), CCL20 (MIP-3a), complement components C3, and C5, 2,3 alpha gal, CCL21 (MIP-2), CCL23 (MPIF-1), CCL24 (MPIF-2/eotaxin-2), CCL25 (TECK), CCL26, CCL3 (MIP-1a), CCL4 (MIP-1b), CCL5 (RANTES), CCL7 (mcp-3), CCL8 (mcp-2), CXCL1, CXCL10 (IP-10), CXCL11 (I-TAC/IP-9), CXCL12 (SDF1), CXCL13, CXCL14, CXCL2, CXCL3, CXCL5 (ENA-78/LIX), CXCL6 (GCP-2), CXCL9, IL13, and IL8. Other types of cells that may be quantified is monocytes. In addition to phenotype, production of cytokines by said monocytes may be assessed. Representative cytokines include: activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, collagen receptors. .alpha..sub.1.beta..sub.1 and .alpha..sub.2.beta..sub.1, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation phingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin and fibronectin receptor.alpha.5.beta.1, Factor X, HB-EGF, HBNF, HGF, HUAF, heart derived inhibitor of vascular cell proliferation, IL1, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-.beta., PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-. beta., and TGF-. beta. receptors, TIMPs, TNF-alpha, transferrin, thrombospondin, urokinase, VEGF-A, VEGF-B, VEGF-C, VEGF-D, VEGF-E, VEGF, VEGF.sub. 164, VEGI, E and G-VEGF. Assessment of these proteins may be performed using various means known in the art. In some embodiments this is performed by protein quantification through plate bound means. Protein assays identify and quantify proteins such as hormones and enzymes, by acting as antigens or antibodies in a chemical reaction. One of the most common protein assays is enzyme-linked immunosorbent assay (ELISA). In a direct ELISA an antigen analyte is adsorbed to a plate and a blocking agent is added to block potential binding sites from non-specific materials. An antibody-enzyme complex is added to bind with the antigen analyte and the plate is washed to remove unbound antibody-enzyme complexes. An appropriate enzyme substrate is added to produce an optical signal proportional to the amount of antigen analyte in the sample. In a Sandwich ELISA, a matched pair of antibodies forms a sandwich structure containing a first outer antibody layer to capture the analyte, an internal layer comprising the antigen analyte and a second outer antibody layer to detect the analyte. The capture antibody is initially bound to the plate and then binds with the antigen analyte contained in a test sample. After washing, a detection antibody-enzyme complex is added to bind with the antigen analyte and the plate is washed to remove unbound capture antibody-enzyme complexes. An appropriate enzyme substrate is added to produce an optical signal proportional to the amount of antigen analyte in the sample. Direct ELISA is faster because only one antibody is being used and fewer steps are required. Sandwich ELISA can have a lower detection limit because each capture antibody can contain several epitopes that can be bound by detection antibodies. Sandwich ELISA can also be made more sensitive using avidin-biotin complexes which have several sites for enzymes to provide multiple enzymes per analyte. This can amplify the detection signal by ten to a few hundred times. In contrast, cell cultures and PCR can produce millions or more copies. Protein assays are relatively easy to use, rapid and low cost. The detection of messenger RNA can also be utilized as a means of quantification of protein production. Various approaches have been employed to quantify nucleic acid analytes using redox assays. Assays that directly detect nucleic acids analytes without amplification claim to match the sensitivity of ELISA. However these techniques lack any substantial benefit for ELISA users to invest the time and cost to adopt a new technological platform. Other approaches have been employed to quantify nucleic acid analytes using redox assays by improving the signal-to-noise ratio. One approach reduces the active surface area of a biosensor working electrode by replacing a conventional solid working electrode with a nanobiosensor comprising randomly distributed forests of nanoscale structures on the electrode surface (Lieber, et al, Thorpe, et al). Another nanobiosensor approach replaces the randomly distributed forests of nanoscale structures with ordered arrays of nanoscale structures spaced at least 1 μm apart to further reduce the surface area of a working electrode (Gordon, et al). These approaches allowed the guanine signal to be better distinguished from noise over conventional solid working electrodes but not to the degree required for direct measurement of the low level of redox species associated with target bio-analytes such as guanine molecules. Fabrication of nanoscale structures, such as 100 nm diameter carbon nanotubes, provides additional complexity over microscale structures and require specialized production equipment with high cost and limited throughput, poor production yields, and high unit costs for nanobiosensors.

there is provided a signal amplification sandwich structure for amplifying, detecting and/or quantifying an analyte or multiple different analytes in a fluid sample, wherein said structure comprises (a) a first outer layer comprising a multifunctional particle conjugated with a plurality of a first analyte binding material for binding the analyte, and the multifunctional particle is also conjugated on its outer structure or filled in its inner structure with a plurality of an electrochemically detectable oligonucleotide tag in greater amounts than said analyte in the inner layer; (b) an inner layer comprising said analyte; and (c) a second outer layer comprising a biosensor working electrode, or a sorbent situated near a biosensor working electrode, conjugated with a plurality of a second analyte binding material for binding said analyte that is a matched pair with the first analyte binding material. The electrochemically detectable oligonucleotide tags are for signal amplification, wherein said oligonucleotide tags are single-stranded, duplex or quadruplex, wherein the majority of nucleotides within said oligonucleotide tags are guanine, wherein the number of guanine per electrochemical detectable oligonucleotide tag ranges from 8 to 400, and when a unique electrochemically detectable oligonucleotide tag is used to amplify, detect and/or quantify said analyte or multiple different analytes said oligonucleotide tag is selected from the group consisting of guanine, adenine, thymine, and cytosine.

The multifunctional particles are for delivering said electrochemically detectable oligonucleotide tags to the analyte and for other functions that enhance analyte amplification, detection and/or quantification, wherein the inner structure of said multifunctional particles is selected from the group consisting of structural materials, magnetic materials, optical materials, nuclear materials, radiological materials, quantum materials, biological materials, energetic materials, electrochemical materials, chemical materials, pharmaceutical materials, antibiotic materials, chemotherapy materials, antibodies, and combinations thereof, wherein the number of electrochemically detectable oligonucleotide tags per multifunctional particle ranges from 102 to 1013, wherein the multifunctional particles are spherical and/or nonspherical, wherein the diameter of spherical multifunctional particles ranges from 0.05 to 400 micrometers, wherein the surface area of nonspherical multifunctional particles has an equivalent surface area of spherical multifunctional particles with ranges from 0.05 to 400 micrometers, and wherein the surface of the multifunctional particles is smooth, rough, porous, or extended with attachments to other particles.

The signal analyte amplification performance of said signal amplification sandwich structure can be tuned to meet the desired limit of detection by adjusting one or more of the following parameters: (a) the number of electrochemically detectable oligonucleotide tags per multifunctional particle; (b) the number of guanines per electrochemically detectable oligonucleotide tag; (c) the size of the multifunctional particle for delivering electrochemically detectable oligonucleotide tags or electrochemical materials; and (d) the surface area of the multifunctional particle for conjugating electrochemically detectable oligonucleotide tags.

The majority of the nucleotides within said quadruplex electrochemically detectable oligonucleotide tags are guanine with at least 4 guanine in a square tetrad structure and an electrochemical detection technique produces 8-oxoguanine signals; wherein the majority of the nucleotides within said quadruplex electrochemically detectable oligonucleotide tags are adenine with at least 4 adenine in a square tetrad structure and an electrochemical detection technique produces 8-oxoadenine signals; wherein the majority of the nucleotides within said quadruplex electrochemically detectable oligonucleotide tags are thymine with at least 4 thymine in a square tetrad structure and an electrochemical detection technique produces 8-oxothymine signals; wherein the majority of the nucleotides within said quadruplex electrochemically detectable oligonucleotide tags are cytosine with at least 4 cytosine in a square tetrad structure and an electrochemical detection technique produces 6-oxocytosine signals; and wherein multiple quadruplexes can form on different segments of the same electrochemically detectable oligonucleotide tag and produce oxo derivative signals from the oxidation of one or more different oxo derivatives.

In accordance with another aspect of the invention, there is also provided a method for amplifying, detecting and/or quantifying one or more analytes in a fluid sample, and diagnosing a disease, outbreak or condition, wherein said method comprises the following steps performed sequentially: (a) providing an artificial intelligence assessment system to recommend actions for assessment of that queries humans, devices, files, records, images, and databases about factors related to diagnosing the disease, outbreak or condition; (b) providing a means for amplifying, detecting and/or quantifying one or more analytes in the fluid sample comprising: i. providing the fluid sample that may contain non-specific materials and an analyte or multiple different analytes; ii. providing one or more sets of multifunctional particle conjugates, wherein each set comprises a plurality of a multifunctional particle conjugated with a plurality of a first analyte binding material and is also conjugated with a plurality of an electrochemically detectable oligonucleotide tag in greater amounts than said analyte to create multifunctional particle-analyte complexes if said analyte or said multiple different analytes are present; iii. providing one or more sets of biosensor working electrodes or one or more sets of sorbents situated near the biosensor working electrodes wherein each biosensor working electrode is associated with said analyte or said group of multiple different analytes that may be present in said sample and wherein each biosensor working electrode or sorbent is conjugated with a plurality of a second analyte binding material that is a matched pair with the first analyte binding material to create signal amplification sandwich structures if said analyte is present; and iv. providing an electrochemical detection technique that produces peak electrochemical signals on each biosensor working electrode in proportion to the quantity of said analyte or said group of multiple different analytes if said analyte or said group of multiple different analytes is present in the fluid sample; (c) providing one or more test results consisting of analyte quantities, and non-bioanalyte and/or bioanalyte levels from other sources that may be associated with the disease, outbreak or condition; (d) providing an artificial intelligence diagnosis system to diagnose and recommend actions for treatment of that interprets said electrochemical signals, non-bioanalyte parameters and/or bioanalyte parameters to diagnosis the disease, outbreak or condition; and (e) providing an artificial intelligence learning system to incorporate improvements, additions and modifications to the artificial intelligence systems and its constituents.

In some embodiments, absolute numbers of monocytes including macrophage precursors, absolute numbers of eosinophils, and/or absolute counts of basophils. Physiological state data may include, without limitation, immune function data such as Interleukine-6 (IL-6), TNF-alpha, systemic inflammatory cytokines, and the like.

Other datapoints may be utilized, for example, without limitation, data describing blood-born lipids, including total cholesterol levels, high-density lipoprotein (HDL) cholesterol levels, low-density lipoprotein (LDL) cholesterol levels, very low-density lipoprotein (VLDL) cholesterol levels, levels of triglycerides, and/or any other quantity of any blood-born lipid or lipid-containing substance. Physiological state data may include measures of glucose metabolism such as fasting glucose levels and/or hemoglobin A1-C(HbA1c) levels. Physiological state data may include, without limitation, one or more measures associated with endocrine function, such as without limitation, quantities of dehydroepiandrosterone (DHEAS), DHEA-Sulfate, quantities of cortisol, ratio of DHEAS to cortisol, quantities of testosterone quantities of estrogen, quantities of growth hormone (GH), insulin-like growth factor 1 (IGF-1), quantities of adipokines such as adiponectin, leptin, and/or ghrelin, quantities of somatostatin, progesterone, or the like. Physiological state data may include measures of estimated glomerular filtration rate (eGFR). Physiological state data may include quantities of C-reactive protein, estradiol, ferritin, folate, homocysteine, prostate-specific Ag, thyroid-stimulating hormone, vitamin D, 25 hydroxy, blood urea nitrogen, creatinine, sodium, potassium, chloride, carbon dioxide, uric acid, albumin, globulin, calcium, phosphorus, alkaline phosphatase, alanine amino transferase, aspartate amino transferase, lactate dehydrogenase (LDH), bilirubin, gamma-glutamyl transferase (GGT), iron, and/or total iron binding capacity (TIBC), or the like. Physiological state data may include antinuclear antibody levels. Physiological state data may include aluminum levels. Physiological state data may include arsenic levels. Physiological state data may include levels of fibrinogen, plasma cystatin C, and/or brain natriuretic peptide.

In some embodiments, body measurements may be related to particular dimensions of the human body. A “dimension of the human body” as used in this disclosure, includes one or more functional body systems that are impaired by disease in a human body and/or animal body. Functional body systems may include one or more body systems recognized as attributing to root causes of disease by functional medicine practitioners and experts. A “root cause” as used in this disclosure, includes any chain of causation describing underlying reasons for a particular disease state and/or medical condition instead of focusing solely on symptomatology reversal. Root cause may include chains of causation developed by functional medicine practices that may focus on disease causation and reversal. For instance and without limitation, a medical condition such as diabetes may include a chain of causation that does not include solely impaired sugar metabolism but that also includes impaired hormone systems including insulin resistance, high cortisol, less than optimal thyroid production, and low sex hormones. Diabetes may include further chains of causation that include inflammation, poor diet, delayed food allergies, leaky gut, oxidative stress, damage to cell membranes, and dysbiosis. Dimensions of the human body may include but are not limited to epigenetics, gut-wall, microbiome, nutrients, genetics, and/or metabolism.

Physiological state data may include genomic data, including deoxyribonucleic acid (DNA) samples and/or sequences, such as without limitation DNA sequences or other genetic sequences contained in one or more chromosomes in human cells. Genomic data may include, without limitation, ribonucleic acid (RNA) samples and/or sequences, such as samples and/or sequences of messenger RNA (mRNA) or the like taken from human cells. Genetic data may include telomere lengths. Genomic data may include epigenetic data including data describing one or more states of methylation of genetic material. Physiological state data may include proteomic data, which as used herein is data describing all proteins produced and/or modified by an organism, colony of organisms, or system of organisms, and/or a subset thereof. Physiological state data may include data concerning a microbiome of a person, which as used herein includes any data describing any microorganism and/or combination of microorganisms living on or within a person, including without limitation biomarkers, genomic data, proteomic data, and/or any other metabolic or biochemical data useful for analysis of the effect of such microorganisms on other physiological state data of a person.

The present invention generally provides structures, methods and devices for amplifying, detecting and/or quantifying an analyte or multiple different analytes in a fluid sample from a single integrated device. This invention allows ultra-low levels of virtually any biological analyte to be detected and quantified rapidly, simply and inexpensively with an electrochemical biosensor using a novel electrochemical signal amplification technique. Electrochemical detection is among the easiest, most rapid and least costly biodetection technique on the market and is the gold standard for quantifying glucose, metabolites, electrolytes, and blood gases. However, its applications are limited to the subset of analytes that have redox properties and also are present in concentrations that are high enough to be detected by an electrochemical biosensor.

The invention amplifies detection signals from low level analytes using an innovative sandwich structure that replaces optical labels with a massive amount of electrochemically detectable oligonucleotide tags rich in electroactive guanine. The tags bind with analytes to amplify the associated detection signal. An electrochemical technique generates a signal in proportion to the guanine level measured at the working electrode which is also proportional to the analyte level in the sample. Selective binding is achieved with matched pairs of either commercial or custom analyte binding materials such as antibodies or DNA probes.

A starting point for describing this invention is a conventional lateral flow immunoassay. These assays typically bind analytes with conjugates comprising a nanoparticle that may be coated or conjugated with an optical label and an antibody for binding with the analyte to form a nanoparticle-analyte complex. The complex is delivered to a sorbent that is coated or conjugated with a capture antibody for capturing the complex and forming a sandwich structure on the sorbent. The optical labels on the sandwiches are then measured with an optical reader or observed visually to determine if a measurable about of optical labels is formed based on the presence of the associated analytes.

As a comparison, this invention replaces conventional immunoassay conjugates with a novel and non-obvious conjugate. The conjugate's nanoparticle is replaced with a multifunctional structure that can be a microparticle or other shape with a much larger surface area than a nanoparticle. This allows orders of magnitude more detection tags to be conjugated to the particle surface to increase the ability of the assay to detect low levels of analytes. The multifunctional particle can also contain a functional material in its interior such as a magnetic material or antimicrobial agent. This provides additional functionality for the conjugate to improve the assay performance such as magnetic separation, or provide functionality beyond the assay such as automatically killing infective pathogens in the test sample to reduce the incidence of transmission.

The invention further replaces conventional immunoassay optical label with a novel and non-obvious electrochemical tag. The tag is an electrochemically detectable oligonucleotide rich in electroactive nucleotides such as guanine or nucleotide derivates such as 8-oxoguanine. Not only is the oligonucleotide low cost and commercially available from multiple suppliers, it can be configured for extremely low limits of detection. This is done by increasing the length of the oligonucleotide to contain more electroactive guanine and/or increase the size of the particle to allow more oligonucleotides to be conjugated to the surface. Another advantage is the different forms of the oligonucleotide provide a different signal peak and a greater amplitude at lower levels. For example a single-stranded oligonucleotide consisting of guanine provides a guanine oxidation peak at around 0.9 V, while a quadruplex oligonucleotide consisting of guanine provides an 8-oxoguanine peak at around 0.47 V. The 8-oxoguanine peak is also more distinguishable at lower levels of tags and analyte, allowing quadruplex oligonucleotide tags to provide lower detection limits that single-stranded oligonucleotides. Another benefit is that because different nucleotides and nucleotide derivatives produce oxidation peaks at different potentials, it is possible to differentiate tags associated with different analytes using the same biosensor working electrode by noticing the potential where the oxidation scan is producing the peak or peaks. This can allow multiplexing at the same electrode. Other examples of how the invention can be used for multiplexing is described below. Another benefit of the oligonucleotide tag over optical labels is that the electrochemical signals are quantitative and are easily measured in a digital format without requiring instruments to transduce the optical signal to electrical. This is seen in electrochemical glucose meters where the electrical signal from the electrochemical reaction is rapidly and easily measured as a quantity that is displayed on a low cost glucose meter and optically communicated though a wireless network to a central database. As an example of an embodiment of the invention, the device units are configured as a lateral flow test cartridge and an instrument comprising a potentiostat such as an EmStat (DropSens BV, Houten, The Netherlands). A liquid sample that may contain E. coli and nonspecific materials is inserted into an inlet of the test cartridge's sample collection unit. The liquid sample flows laterally to the signal amplification tag attachment unit which contains a set of multifunctional particle conjugates for amplifying the signal. Each conjugate comprises a multifunctional particle conjugated with a plurality of a polyclonal antibody for binding E. coli and a plurality of an electrochemically detectable oligonucleotide tag in much greater amounts than the E. coli being bound to the conjugate. E. coli in the sample bind with the conjugates and multifunctional particle-E. coli complexes. The complexes flow laterally to the signal amplification capture unit which contains biosensor working electrodes with monoclonal antibodies conjugated on or near the working electrode surfaces for capturing the multifunctional particle-E. coli complexes. E. coli in the complexes bind with capture unit's monoclonal antibodies to form signal amplification sandwich structures. The test cartridge is inserted into the analyzer which applies a square wave voltammetry scan and produces an electrical current signal proportional to the electrochemically detectable tags bound in signal amplification sandwich structures at the biosensor working electrode. The signal is also proportional to the concentration of E. coli in the sample. The E. coli concentration is determined from a preprogrammed mathematical formula that converts the peak electrical current form the scan to a concentration based on peak electrical currents of known samples. A second working electrode is used as a negative control to verify that the test is valid. A benefit of this invention is that the method for measuring analytes uses as few as 3 steps which are very simple and can be automated. This allows the method to be portable and used in diverse platforms including lateral flow devices, 96-well microtiters, high throughput systems, inline systems, and other common assay platforms. Another benefit is that the method is rapid as each step can be conducted in minutes. Another benefit is that the method employs a small number of low cost reagents and off the shelf instrument components, making the cost per test very low. Similar benefits are also provided with lateral flow devices using conventional optical tags.

Outcomes from the predictive test can be utilized to guide therapeutic administration of cell therapies, conventional therapies, and cytokine therapies. In one embodiment administration of mesenchymal stem cells is performed. Said mesenchymal stem cells may be naturally occurring mesenchymal stem cells, or mesenchymal stem cells derived from pluripotent sources.

In one embodiment the present invention provides transformed immune cells generated in a donor specific manner using pluripotent stem cell generating technologies, such as inducible pluripotent stem cell technology. These technologies are combined with transfection of staid DC or DC-like cell with toleogenic molecules. Furthermore, iPSC and/or derivatives of said iPSC differentiated into the DC lineage may be engineered in a manner that exhibit a gene specific targeted knock-out phenotype. Such transformed immune cells can be used in a variety of therapeutic in vitro, ex vivo and in vivo methods to modulate T cell activity and thus have use in therapeutic approaches for the treatment of immune disorders in mammalian subjects. he immune cells of the invention exhibit a targeted gene-specific knockout phenotype which may be accomplished using any technique that provides for the targeted silencing of an endogenous gene. In one aspect of the invention the technique of RNAi (RNA interference) was used to create transformed immune cells suitable for use for the modulation of T cell activity in vitro, ex vivo or in vivo. In this aspect, the immune cells are transfected with a siRNA (small interfering RNA) designed to target and thus to degrade a desired mRNA in order not to express the encoded protein that is involved in T cell activity. Thus such transfected immune cells may be used to suppress or stimulate immune system functioning via the modulation of T cell activity. It is understood by those of skill in the art that any method for silencing a specific gene may be used in the present invention. Representative examples of suitable techniques include but are not limited to RNAi and hybrid DNA/RNA constructs. The hybrid DNA/RNA constructs are essentially siRNA constructs in which the nucleic acid composition used for silencing is altered to include DNA

Immune cells for use in the present invention may be selected from antigen presenting cells (APC) and endothelial cells. Both APC and endothelial cells are known to be able to activate T cells, or in the case preferred by the inventors in the present invention, to induce T cell death, T cell anergy, or Treg differentiation. In preferred embodiments of the invention, the immune cells are APC that may be selected from the group consisting of macrophages, myeloid cells, B lymphocytes, DC and mixtures thereof. It is also within the scope of the present invention to use other APC capable of inducing T cell inhibition through the T cell receptor as is understood by one of skill in the art. In particularly preferred embodiments of the invention, the immune cell is a DC. APC such as DC are known to be phagocytic in nature and thus tend to take up molecules within their environment. In the present invention DC is specifically demonstrated to be successfully altered with siRNA to exhibit a stable phenotype. Therefore one of skill in the art would readily understand that any APC may be altered in accordance with the present invention and used in the methods of the invention. It is also understood that a combination of different types of immune cells may be used in the methods of the present invention. According to an embodiment of the invention, DC are transformed with a designed siRNA. In this embodiment DC must be isolated from a subject and expanded in vitro. DC are typically derived from a source such as bone marrow, peripheral blood, spleen and lymph. In a preferred embodiment DC are generated from iPSC cells. Generation of DCs from iPSC is known in the art and various means disclosed previously may be utilized. In one embodiment, the means described below are utilized. Induced pluripotent stem cells and methods of producing them are utilized as known in the art. A method for inducing pluripotency of differentiated cells, such as somatic cells, was first disclosed by Yamanaka (WO 2007/069666). In this method, somatic cells are reprogrammed using three main nuclear reprogramming factors, namely an Oct family gene, a K1f family gene and a Sox family gene (preferably Sox2). The factors are preferably Oct3/4, K1f4 and Sox2. A fourth reprogramming factor, namely the product of a Myc family gene (preferably c-Myc), may also be used. Numerous different methods have since been disclosed for inducing pluripotency in somatic cells. In some embodiments, various are performed such as culture of the “mother cells” in the presence of a histone deacetylase inhibitor. Suitable histone deacetylase inhibitors include valproic acid, sodium phenylbutyrate, trichostatin A, sulforaphane, and phenylbutyrate.

For the purpose of the invention, the generated iPSCs cells typically display the characteristic morphology of human embryonic stem cells (hESCs), express the pluripotency-associated markers SSEA-4 and TRA1-60, the transcription factors Oct-4 and Nanog and differentiate in vitro into cell types derived from each of the three embryonic germ layers. The iPSCs may be an established cell line. More preferably, the iPSCs are produced from somatic cells taken from a patient to be treated in accordance with the invention. The iPSCs may be derived from any human somatic cell. Suitable cells include, but are not limited to, keratinocytes, dermal fibroblasts or leukocytes derived from peripheral blood. The iPSCs are preferably derived from dermal fibroblasts. Techniques for culturing iPSCs are well known to a person skilled in the art. Suitable conditions are discussed above. Conditions suitable for inducing stem cells to differentiate into DCs are known in the art. For instance, suitable conditions are disclosed in Tseng, S-Y. et al. Regen. Med. 4, 513-526 (2009). However, it is surprising that culturing human iPSCs under these condition results in DCs that are capable of cross presenting an antigen to naïve CD8+ T lymphocytes.

In a preferred embodiment, the method comprises (a) culturing the iPSCs in a medium comprising granulocyte macrophage-colony stimulating factor (GM-CSF) for sufficient time to produce monocytic cells, (b) culturing the monocytic cells under conditions that induce the formation of immature dendritic cells and (c) culturing the immature dendritic cells in a medium comprising growth factors that induce maturation of the dendritic cells. The sufficient time in step (a) is typically from 13 to 17 days. In step (a), the medium preferably further comprises one or more of stem cell factor (SCF), vascular endothelial growth factor (VEGF) and bone morphogenic protein (BMP-4). The medium more preferably initially comprises all three of SCF, VEGF and BMP-4 and each is successively removed. Step (a) most preferably comprises initially culturing the iPSCs in a medium comprising GM-CSF, SCF, VEGF and BMP-4, removing BMP-4 from day 5 onwards, removing VEGF from day 14 onwards and removing SCF from day 19 onwards until monocytic cells are produced. The sufficient time in step (b) is typically from 9 to 15 days. Suitable conditions for forming immature DCs from monocytic cells are known in the art. Step (b) preferably involves culturing the monocytic cells in a medium comprising GM-CSF and interleukin-4 (IL-4) for sufficient time to produce immature DCs. Step (c) takes from 36 hours to 4 days, preferably about 2 days (48 hours). The medium in step (c) preferably comprises GM-CSF, tumor necrosis factor-α (TNFα), prostaglandin-E2 (PGE2), interleukin-1β (IL-1β) and interferon-γ (IFNγ). Steps (a) to (c) typically take from 21 to 32 days. Preferred concentrations of the various growth factors are as follows: GM-CSF—from 25 to 75 ng/ml, more preferably 50 ng/ml; SCF—from 10 to 30 ng/ml, more preferably 20 ng/ml; VEGF—from 25 to 75 ng/ml, more preferably 50 ng/ml; BMP-4—from 25 to 75 ng/ml, more preferably 50 ng/ml; IL-4—from 10 to 150 ng/ml, more preferably 25 or 100 ng/ml; TNFα—from 10 to 30 ng/ml, more preferably 20 ng/ml; PGE2—from 0.5 to 1.5 ng/ml, more preferably 1.0 ng/ml; IL-1β—from 5 to 15 ng/ml, more preferably 10 ng/ml; and IFNγ—from 10 to 20 ng/ml, more preferably 15 ng/ml. The growth factors used in the method of the invention are typically the human forms. The growth factors used in the method of the invention are typically recombinant. The use of such factors means that the DCs of the invention are produced in clinically relevant conditions, i.e. in the absence of trace amounts of endotoxins and other environmental contaminants, such as lipopolysaccharides, lipopeptides and peptidoglycans, etc. This makes the DCs of the invention particularly suitable for administration to patients. The method preferably further comprises isolating the DCs of the invention. Any of the methods discussed above may be used. The invention also provides a method for producing a population of the invention that is suitable for administration to a patient, wherein the method comprises producing iPSCs from somatic cells obtained from the patient and producing a population of the invention from those iPSCs using the method of the invention described above. The population will be autologous with the patient and therefore will not be rejected upon implantation. The invention also provides a population of the invention that is suitable for administration to a patient and is produced in this manner. Alternatively, the invention provides a method for producing a population of the invention that is suitable for administration to a patient, wherein the method comprises the differentiation of partially-matched iPSCs obtained from a public bank of clinically-approved lines. Substances which stimulate hematopoiesis (i.e. G-CSF and GM-CSF) may be first administered to the subject in order to increase the number of DC. Blood is treated to isolate the DC from other cell types by standard methods known in the art. Isolated DC cultured in vitro may be treated with cytokines to increase their number. Methods for isolating and ex vivo culture of DC are known in the art and described for example in U.S. Pat. Nos. 5,199,942, 5,851,756, 6,017,527, 6,251,665, 6,458,585 and 6,475,483 (the disclosures of which are incorporated herein by reference in their entirety). The present invention also encompasses therapeutic methods for the treatment of a variety of immune disorders in a mammalian subject. The methods may involve the use of a siRNA designed for use directly in vivo to block the expression of a gene by an immune cell, the gene expressing a protein involved in the activity of T cells which elicits an immune disorder. Alternatively, the methods may involve the use of an immune cell which contains at least one double-stranded RNA molecule (siRNA) that inhibits the expression of an endogenous target gene encoding a surface marker, a chemokine, a cytokine, an enzyme or a transcriptional factor. In preferred embodiments of the invention, the methods of the invention comprise the use of an altered (i.e. transformed) DC that contains a double-stranded RNA molecule that inhibits the expression of an endogenous target gene encoding a surface marker, a chemokine, a cytokine, an enzyme or a transcriptional factor. Still in other embodiments, the therapeutic method may involve ex vivo treatment of tissues and/or organs intended for transplantation. In aspects of the invention, the siRNA possesses specific homology to part or to the entire exon region of a surface marker, a chemokine, a cytokine, an enzyme or a transcriptional factor normally expressed by the immune cell such that the gene is silenced It is understood by one of skill in the art that the siRNA as herein described may also include altered siRNA that is a hybrid DNA/RNA construct or any equivalent thereof. In preferred embodiments of the invention the transfected DC cells are prepared by the method of RNAi. RNA interference is a mechanism of post-transcriptional gene silencing. Specific gene silencing is mediated by short strands of duplex RNA of approximately 21 nucleotides in length (termed small interfering RNA or siRNA) that target the cognate mRNA sequence for degradation. While many techniques have been used to block specific molecules in vitro and in vivo, such as anti-sense oligonucleotides (Gerwitz, A. M. 1999. Curr Opin Mol Ther 1:297) and monoclonal antibodies (Drewe, E., et al., 2002. J Clin Pathol 55:81), RNAi was used in the present invention because it provides several distinct advantages. First, mRNA degradation by siRNA is extremely efficient as only a few copies of dsRNA are necessary to activate the RNA Induced silencing complex (RISC) (Martinez, J. A. et al., 2002. Cell 10:563). Once RISC is activated it can conduct multiple rounds of gene-specific mRNA cleavage. Second, RNAi is specific, in that only sequences with identity to one of the strands of dsRNA will be cleaved (Hannon, G. J. 2002. Nature 418:244). Third, the RNAi effect is long lasting and can be spread to progeny cells after replication, although a dilution effect is evident in mammalian cells (Fire, A., et al., 1998. Nature 391:806). This technique is relatively simple, giving rise to an in vitro knock down phenotype within days that can be confirmed with many antibody based detection systems (such as ELISA or Western Blotting), or if an antibody is not available, by RT-PCR or functional assays. DC may be transformed with siRNA alone, siRNA contained within a plasmid or vector that results in the production of the siRNA, siRNA contained within a plasmid or vector that further expresses a selected antigen and siRNA together with a mRNA from a tumor cell. In the case of the plasmid or is vector further expressing a selected antigen, the DC will process or modify the antigen in a manner-to promote the stimulation of T cell activity by the processed or modified antigens. Methods for making siRNA and cell transformation are described for example in U.S. Patent Application 2002/0173478, U.S. Patent Application 2002/0162126, PCT/US01/10188, PCT/EP01/13968 and in Simeoni F., et al., 2003 Nucleic Acids Res June 1;31 (11): 2717-24 (the disclosures of which are incorporated herein in their entirety). Methods for producing antigen pulsed DC are known and exemplified for example in U.S. Pat. No. 6,497,876 and U.S. Pat. No. 6,479,286 (the disclosures of which are incorporated herein by reference in their entirety). Methods for making siRNA plasmids or vectors are also known and described for example in U.S. Patent Application 2003/0104401, in Morris M. C., et al., 1997. Nucleic Acid Res. July 15:25(14):2730-6 and in Van De Wetering M., et al., 2003, EMBO June;4(6):609-15 (the disclosures of which are incorporated herein in their entirety). Suitable lipid-based vectors may include but are not limited to lipofectamine, lipofectin, oligofectamine and GenePorter™. Methods for producing tumor derived RNA for pulsing DC are also known to those of skill in the art and are described for example in U.S. Patent Application 2002/0018769 (the disclosure of which is incorporated herein in its entirety). In embodiments of the invention, DC are transformed to contain a double-stranded RNA molecule that inhibits the expression of an endogenous target gene encoding a protein that either suppresses T cell activation or alternatively stimulates T cell activation. For the suppression of T cell activation, the immune cells of the invention are transformed with a double-stranded RNA molecule that inhibits the expression of a gene that encodes a co-stimulatory molecule, cytokine, adhesion molecule, enzyme or transcription factor. Representative examples of such co-stimulatory molecules, cytokines, adhesion molecules, enzymes and transcription factors may be selected from the group consisting of TNFα, IL-1, IL-1b, IL-2, TNFβ, IL-6, IL-7, IL-8, IL-23, IL-15, IL18, IL-12, IFNγ, IFNα, lymphotoxin, DEC-25, CD11c, CD40, CD80, CD86, MHCI, MHCII, ICAM-1, TRANCE, CD200, CD200 receptor, CD83, CD2, CD44, CD91, TLR-4, TLR-9, 4-1 BBL, nicotinic receptor, GITR-L, OX-40L, CD-CK1, TARC/CCL17, CCL3, CCL4, CXCL9, CXCL10, IKK-β, NF-κB, STAT4, ICSBP/IFN, regulatory factor 8, TRAIL, Inos, arginase, FcgammaRI and II, thrombin, MIP-1α and MIP-1B.

In some embodiments of the invention, enhancement of mesenchymal stem cell activation is mediated by stimulation of T regulatory cells in vivo, which is accomplished by administration of Aldesleukin (Proleukin, Novartis), which is a commercially available IL-2 licensed for the treatment of metastatic renal cell carcinoma in the UK. It is produced by recombinant DNA technology using an Escherichia coli strain, which contains a genetically engineered modification of the human IL-2 gene, and is administered either intravenously or subcutaneously (SC). Following short intervenous infusion, its pharmacokinetic profile is typified by high plasma concentrations, rapid distribution into the extravascular space and a rapid renal clearance. The recommended doses for continuous infusion and subcutaneous injection (as detailed in the Summary of Product Characteristics) are repeated cycles of 18×106 IU per m2 per 24 hours for 5 days and repeated doses of 18×106 IU, respectively. Peak plasma levels are reached in 2-6 hours after SC administration, with bioavailability of aldesleukin ranging between 31% and 47%. The process of absorption and elimination of subcutaneous aldesleukin is described by a one-compartment model, with a 45 min absorption half-life and an elimination half-life of 3-5 hours. Natural IL-2 was first identified in 1976 as a growth factor for T lymphocytes. It is produced by human cluster designation (CD) 4+ and some CD8+ T-cells and is synthesized mainly by activated T-cells, in particular CD4.sup.+ helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilit: ites the proliferation and differentiation of B-cells and the synthesis of immunoglobulin by B-cells, and stimulates the generation, proliferation and activation of natural killer (NK). IL-2 is known to play a central role in the generation of immune responses. In cancer clinical trials, high-dose recombinant IL-2 (e.g., IV bolus dose of 600,000 international units (IU)/kg every 8 hours for up to 14 doses) demonstrated antitumor activity in metastatic renal cell carcinoma (RCC) and metastatic melanoma. Accordingly, such high-dose IL-2 was approved for the treatment of metastatic RCC in Europe in 1989 and in the US in 1992. In 1998, approval was obtained to treat patients with metastatic melanoma. Recombinant human IL-2 (Aldesleukin) (Proleukin®-Novartis Inc. & Prometheus Labs Inc.) is currently approved by the United States Food and Drug Administration (US FDA). However, IL-2 has a dual function in the immune response in that it not only mediates expansion and activity of effector cells, but also is crucially involved in maintaining peripheral immune tolerance. A major mechanism underlying peripheral self-tolerance is IL-2 induced activation-induced cell death (AICD) in T cells. AICD is a process by which fully activated T cells undergo programmed cell death through engagement of cell surface-expressed death receptors such as CD95 (also known as Fas) or the TNF receptor. When antigen-activated T cells expressing a high-affinity IL-2 receptor (after previous exposure to IL-2) during proliferation are re-stimulated with antigen via the T cell receptor (TCR)/CD 3 complex, the expression of Fas ligand (FasL) and/or tumor necrosis factor (TNF) is induced, making the cells susceptible for Fas-mediated apoptosis. This process is IL-2 dependent and mediated via STAT5. By the process of AICD in T lymphocytes tolerance can not only be established to self-antigens, but also to persistent antigens that are clearly not part of the host's makeup, such as tumor antigens.

The invention teaches means of overcoming existing hurdles in the area of detection and treatment of radiological, biological and chemical threats. For example, it is generally accepted in the field of toxicological sciences that conventional therapeutics are utilized based on human design, high-throughput screening, and/or natural substances may be inefficient, riven with noise, limited in application, not efficacious, dangerous or poisonous, and/or not defensible.

Further, in some instances, there are instances of certain pathologies caused by noxious agents that do not have a corresponding existing therapeutic to treat the certain diseases or which provide temporary results against which the disease is refractory. One reason for the lack of an existing therapeutic may be the conventional drug discovery techniques are incapable of discovering the therapeutic needed to treat the certain diseases. By “treat,” we mean that the disease at hand is cured inter alia, that it is not refractory to treatment. The amount of knowledge, data, assumptions, and queries used to discover a therapeutic to treat the certain disease may be unattainable, overwhelming, and/or inefficiently determined, such that conventional drug discovery techniques cannot overcome these obstacles. Improvement is desired in the field of therapeutics.

Through the integration of stem cells and artificial intelligence technologies, such as supervised and unsupervised deep learning, the invention provides means of rapidly scanning databases, as well as utilizing PCA and other forms of analysis to predict which existing compounds may modulate desired activities in conditions of pathology such as those inflicted by chemical, biological and radiological toxins, as well as how to design around existing compounds that provide a signal of efficacy but may have some other properties that are not desirable.

In some embodiments of the invention, various immunological and regenerative cells are utilized as a means of potential treatment. In one embodiment the artificial intelligence algorithm is used for screening of therapeutic approaches. Accordingly, aspects of the present disclosure generally relate to an artificial intelligence engine for generating candidate drugs for biological manipulation. By using various encoding types that enable performing searches in the design space in an efficient manner, the artificial intelligence engine (AI) may enlarge the design space to include the combination of drug information (e.g., structural, physical, semantic, activity, sequence, chemical, etc.). The architecture of the AI engine may include various computational techniques that reduce the computational complexity of using a large design space, thereby saving computing resources (e.g., reducing computing time, reducing processing resources, reducing memory resources, etc.). At the same time, the disclosed architecture may generate superior candidate drugs that include desirable features (e.g., structure, semantics, activity, sequence, clinical outcomes, etc.) found in the larger design space as compared to conventional techniques using the smaller design space. The artificial intelligence (AI) engine may use a combination of rational algorithmic discovery and machine learning models (e.g., generative deep learning methods) to produce enhanced therapeutics that may treat any suitable target disease and/or medical condition. The AI engine may discover, translate, design, generate, create, develop, formulate, classify, and/or test candidate drug compounds that exhibit desired activity (e.g., antimicrobial, immunomodulatory, cytotoxic, neuromodulatory, etc.) in design spaces for target diseases and/or medical conditions. Such candidate drug compounds that exhibit desired activity in a design space may effectively treat the disease and/or medical condition associated with that design space. In some embodiments, a selected candidate drug compound that effectively treats the disease and/or medical condition may be formulated into an actual drug for administration and may be tested in a lab and/or at a clinical stage. In general, the disclosed embodiments may enable rationally discovery of drug compounds for a larger design space at a larger scale, higher accuracy, and/or higher efficiency than conventional techniques. The AI engine may use various machine learning models to discover, translate, design, generate, create, develop, formulate, classify, and/or test candidate drug compounds. Each of the various machine learning models may perform certain specific operations. The types of machine learning models may include various neural networks that perform deep learning, computational biology, and/or algorithmic discovery. Examples of such neural networks may include generative adversarial networks, recurrent neural networks, convolutional neural networks, fully connected neural networks, etc., as described further below; and such networks may also additionally employ methods of or incorporating causal inference, including counterfactuals, in the process of discovery.

In some embodiments, a biological context representation of a set of biological/radiological/chemical threats may be generated. The biological context representation may be a continuous representation of a biological setting that is updated as knowledge is acquired and/or data is updated. The biological context representation may be stored in a first data structure having a format (e.g., a knowledge graph) that includes both various nodes pertaining to health artifacts and various relationships connecting the nodes. The nodes and relationships may form logical structures having subjects and predicates. For example, one logical structure between two nodes having a relation may be “Genes are associated with Diseases” where “Genes” and “Diseases” are the subjects of the logical structure and “are associated with” is the relation. In such a way, the knowledge graph may encompass actual knowledge, rather than simply statistical inferences, pertaining to a biological setting. The information in the knowledge graph may be continuously or periodically updated and the information may be received from various sources curated by the AI engine. The knowledge in the biological context representation goes well beyond “dumb” data that just includes quantities of a value because the knowledge represents the relationships between or among numerous different types of data, as well as any or all of direct, indirect, causal, counterfactual or inferred relationships. In some embodiments, the biological context representation may not be stored, and instead, based on the stream of knowledge included in the biological context representation, may be streamed from data sources into the AI engine that generates the machine learning models. The biological context representation may be used to generate candidate drug compounds by translating the first data format to a second data structure having a second format (e.g., a vector). The second format may be more computationally efficient and/or suitable for generating candidate drug compounds that include sequences of ingredients that provide desired activity in a design space. “Ingredients” as used herein may refer, without limitation, to substances, compounds, elements, activities (such as the application or removal of electrical charge or a magnetic field for a specific maximum, minimum or discrete amount of time), and mixtures. Further, the second format may enable generating views of the levels of activity provided by the sequence of ingredients in a certain design space, as described further below.

At a high level, the AI engine may include at least one machine learning model that is trained to use causal inference to generate candidate drug compounds. One of the challenges with discovering new therapeutics may include determining whether certain ingredients are causal agents with respect to certain activity in a design space. The sheer number of possible sequences of ingredients may be extraordinarily large due to mathematical combinatorics, such that identifying a cause and effect relationship between ingredients and activity may be impossible or, at best, extremely unlikely, to identify without the disclosed embodiments. Based on advances in computing hardware (e.g., graphic processing unit processing cores) and the AI techniques using causal inference described herein, the disclosed embodiments may enable the efficient solving of the task of generating candidate drug compounds at scale. By simulating numerous alternative scenarios to further optimize and hone the accuracy of a sequence of ingredients in the candidate drug compounds, such techniques may enable reducing the number of viable candidate drug compounds. As a result, the embodiments may provide technical benefits, such as reducing resources consumed (e.g., processing, memory, network bandwidth) by reducing a number of candidate drug compounds that may be considered for classification as a selected candidate drug compound by another machine learning model. In some embodiments, one application for the AI engine to design, discover, develop, formulate, create, and/or test candidate drug compounds may pertain to peptide therapeutics. A peptide may refer to a compound consisting of two or more amino acids linked in a chain. Example peptides may include dipeptides, tripeptides, tetrapeptides, etc. Aa polypeptide may refer to a long, continuous, and unbranched peptide chain. Peptides may be simple to manufacture at discovery scale, include drug-like characteristics of small molecules, include safety and high specificity of biologics, and/or provide greater administration flexibility than some other biologics.

Compounds may be tested in various stem cell differentiation assays as well as stem cell proliferation assays. The utilization of stem cells may involve hematopoietic stem cells, which typically express markers such as the adhesion molecule CD34, or other ones such as CD133 and/or CD105. In some cases modulation of marker expression is desired by the drugs being screened. For example, in some embodiments it is desired to increase homing of stem cells to a desired location, so increased CXCR4 is desired.

The disclosed techniques provide numerous benefits over conventional techniques for designing, developing, and/or testing candidate drug compounds for assessment in modulation of biological systems, in some embodiments for modulation of stem cell activity. For example, the AI engine may efficiently use a biological context representation of a set of drug compounds and one or more machine learning models to generate a set of candidate drug compounds and classify one of the set of candidate drug compounds as a selected candidate drug compound. Some embodiments may use causal inference to remove one or more potential candidate drug compounds from classification, thereby reducing the computational complexity and processing burden of classifying a selected candidate drug compound. In addition, benchmark analysis may be performed for each type of machine learning model that generates candidate drugs. The benchmark analysis may score various parameters of the machine learning models that generate the candidate drugs. The various parameters may refer to candidate drug novelty, candidate drug uniqueness, candidate drug similarity, candidate drug validity, etc. The scores may be used to recursively tune the machine learning models over time to cause one or more of the parameters to increase for the machine learning models. In some embodiments, some of the machine learning models may vary in their effectiveness as it pertains to some of the parameters. In addition, to generate subsequent candidate drug candidates, the benchmark analysis may score the candidate drug candidates generated by the machine learning models, rank the machine learning models that generate the highest scoring candidate drug candidates, and/or select the machine learning models producing the highest scoring candidate drug candidates.

Using causal inference, a generative adversarial network (GAN) may be used to generate a set of candidate drug compounds. A GAN refers to a class of deep learning algorithms including two neural networks, a generator and a discriminator, that both compete with one another to achieve a goal. For example, regarding candidate drug compound generation, the generator goal may include generating candidate drug compounds, including compatible/incompatible sequences of ingredients, and effective/ineffective sequences of ingredients, etc. that the discriminator classifies as feasible candidate drug compounds, including compatible and effective sequences of ingredients that may produce desired activity levels for a design space. In one embodiment, the generator may use causal inference, including counterfactuals, to calculate numerous alternative scenarios that indicate whether a certain result (e.g., activity level) still follows when any element or aspect of a sequence changes.

In the practice of the invention, detection of radiological, chemical or biological threats may be performed using a neural network based on Markov models (e.g., Deep Markov Models), which may perform causal inference. In some embodiments, one or more of the counterfactuals used during the causal inference may be determined and provided by the scientist module. The discriminator goal may include distinguishing candidate drug compounds which include undesirable sequences of ingredients from candidate drug compounds which include desirable sequences of ingredients. In some embodiments, the generator initially generates candidate drug compounds and continues to generate better candidate drug compounds after each iteration until the generator eventually begins to generate candidate drug compounds that are valid drug compounds which produce certain levels of activity within a design space.

The discriminator may use a gradient of an objective function to increase the value of the output. The discriminator may be trained as an unsupervised “density estimator,” i.e., a contrast function produces a low value for desired data (e.g., candidate drug compounds that include sequences producing desired levels of certain types of activity in a design space) and higher output for undesired data (e.g., candidate drug compounds that include sequences producing undesirable levels of certain types of activity in a design space). The generator may receive the gradient of the discriminator with respect to each modified candidate drug compound it produces. The generator uses the gradient to train itself to produce modified candidate drug compounds that the discriminator determines include sequences producing desired levels of certain types of activity in a design space. Recurrent neural networks include the functionality, in the context of a hidden layer, to process information sequences and store information about previous computations. As such, recurrent neural networks may have or exhibit a “memory.” Recurrent neural networks may include connections between nodes that form a directed graph along a temporal sequence. Keeping and analyzing information about previous states enables recurrent neural networks to process sequences of inputs to recognize patterns (e.g., such as sequences of ingredients and correlations with certain types of activity level). Recurrent neural networks may be similar to Markov chains. For example, Markov chains may refer to stochastic models describing sequences of possible events in which the probability of any given event depends only on the state information contained in the previous event. Thus, Markov chains also use an internal memory to store at least the state of the previous event. These models may be useful in determining causal inference, such as whether an event at a current node changes as a result of the state of a previous node changing.

The set of candidate drug compounds generated may be input into another machine learning model 132 trained to classify of the set of candidate drug compounds as a selected candidate drug compound. The classifier may be trained to rank the set of candidate drug compounds using any suitable ranking (i.e., for example, non-parametric) technique. For example, in some embodiments, one or more clustering techniques may be used to cluster the set of candidate drug compounds. To classify the selected candidate drug compound, the machine learning model may also perform objective optimization techniques while clustering. To classify the selected candidate drug compound having desired levels of certain types of activity, the objective optimization may include using a minimization and/or maximization function for each candidate drug compound in the clusters. A cluster may refer to a group of data objects similar to one another within the same cluster, but dissimilar to the objects in the other clusters. Cluster analysis may be used to classify the data into relative groups (clusters). One example of clustering may include K-means clustering where “K” defines the number of clusters. Performing K-means clustering may comprise specifying the number of clusters, specifying the cluster seeds, assigning each point to a centroid, and adjusting the centroid. Additional clustering techniques may include hierarchical clustering and density based spatial clustering. Hierarchy clustering may be used to identify the groups in the set of candidate drug compounds where there is no set number of clusters to be generated. As a result, a tree-based representation of the objects in the various groups may be generated. Density-based spatial clustering may be used to identify clusters of any shape in a dataset having noise and outliers. This form of clustering also does not require specifying the number of clusters to be generated.

1. A method of identifying the potential of developing pathogenic radiological, chemical, or biological reactions using a machine learning system programmed to observe, analyze, and predict based on patient-derived datapoints.

2. The method of claim 1, wherein said patient-derived datapoint is perturbation in homeostatic functions.

3. The method of claim 1, wherein said patient-derived datapoint is perturbation in immunological functions.

4. The method of claim 3, wherein said immunological function alteration is assessed by levels of immune modulatory proteins in one or more biological matrices.

5. The method of claim 4, wherein said immune modulatory protein is an aggregation of proteins.

6. The method of claim 5, wherein said aggregation of proteins are exosomes.

7. The method of claim 1, wherein said detection of potential reactions is accomplished by obtaining a biological fluid and irradiating said fluid, exciting components to emit IR radiation in a wavelength range 8-12 μM; providing a detection system with a detector and analyzer to compare IR radiation patterns with a reference database for identifying threats.

8. The method of claim 1, wherein said machine learning system utilizes a knowledge acquisition input platform connected with a database comprising a correlation algorithm to score pathological outcomes based on biological parameters.

9. The method of claim 8, wherein elevation of C-reactive protein more than 25% above baseline is utilized to alert need for increased intensity of monitoring.

10. A method of utilizing one or more artificial intelligence algorithms to suggest therapeutic doses of hematopoiesis-protective therapeutics after exposure to radiation.

11. The method of claim 10, wherein historical data is combined with genotypic, phenotypic, and physiological data in respect to the patient being analyzed.

12. The method of claim 11, wherein gene polymorphisms for Bcl-2Xs are evaluated and incorporated into said calculation, wherein enhanced Bcl-2Xs is associated with enhanced need for mesenchymal stem cell administration.

13. The method of claims 12, wherein said mesenchymal stem cell is derived from a pluripotent stem cell source.

14. The method of claim 13, wherein said mesenchymal stem cell is pretreated with one or more agents capable of augmenting expression of interleukin-10.

15. The method of claim 13, wherein said agent capable of increasing expression of interleukin-10 is brain-derived neurotrophic growth factor.

16. A computer-implemented method for identifying risk of biological, chemical, or radiological threats, categorizing potential risks, and providing treatment recommendations through the utilization of an artificial intelligence system.

17. The method of claim 16, which creates a score for each of the one or more pathologies, reflecting the likelihood and urgency of treatment needed.

18. The method of claim 16, further comprising indicating whether the one or more molecular pathways have synergy in the treatment of the patient.

19. The method of claim 16, wherein said molecular pathways include toll-like receptor-associated pathways.

20. The method of claim 19, wherein said pluripotency-associated gene is the octamer-binding transcription factor 4 (OCT4).
